SEQUENCE LISTING (42 sequences)

SEQ ID NO: 1 - protein, 32 aa, Listeria monocytogenes
KENSISSMAPPASPPASPKTPIEKKHADEIDK

SEQ ID NO: 2 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 4 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 6 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 7 - protein, 14 aa, Listeria monocytogenes
KTEEQPSEVNTGPR

SEQ ID NO: 8 - protein, 28 aa, Listeria monocytogenes
KASVTDTSEGDLDSSMQSADESTPQPLK

SEQ ID NO: 9 - protein, 20 aa, Listeria monocytogenes
KNEEVNASDFPPPPTDEELR

SEQ ID NO: 10 - protein, 33 aa, Listeria monocytogenes
RGGIPTSEEFSSLNSGDFTDDENSETTEEEIDR

SEQ ID NO: 11 - protein, 17 aa, Streptococcus pyogenes
KQNTASTETTTTNEQPK

SEQ ID NO: 12 - protein, 17 aa, Streptococcus equisimilis
KQNTANTETTTTNEQPK

SEQ ID NO: 13 - protein, 98 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 14 - protein, 105 aa, Artificial Sequence
  E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 15 - protein, 158 aa, Artificial Sequence
  E6 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 16 - protein, 158 aa, Artificial Sequence
  E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - DNA, 22 bp, Artificial Sequence
  Primer
ggctcgagcatggagatacacc

SEQ ID NO: 18 - DNA, 28 bp, Artificial Sequence
  Primer
ggggactagtttatggtttctgagaaca

SEQ ID NO: 19 - DNA, 31 bp, Artificial Sequence
  Primer
gggggctagccctcctttgattagtatattc

SEQ ID NO: 20 - DNA, 28 bp, Artificial Sequence
  Primer
ctccctcgagatcataatttacttcatc

SEQ ID NO: 21 - DNA, 27 bp, Artificial Sequence
  Primer
cccgtcgaccagctcttcttggtgaag

SEQ ID NO: 22 - DNA, 25 bp, Artificial Sequence
  Primer
gcggatcccatggagatacacctac

SEQ ID NO: 23 - DNA, 22 bp, Artificial Sequence
  Primer
gctctagattatggtttctgag

SEQ ID NO: 24 - protein, 9 aa, Artificial Sequence
  E7 peptide
RAHYNIVTF

SEQ ID NO: 25 - protein, 9 aa, Artificial Sequence
  cytotoxic T-cell (CTL) epitope for HIV-GAG
AMQMLKETI

SEQ ID NO: 26 - protein, 9 aa, Artificial Sequence
  cytotoxic T-cell (CTL) epitope for Listeria LLO
GYKDGNEYI

SEQ ID NO: 27 - DNA, 55 bp, Artificial Sequence
  Primer
gactacaaggacgatgaccgacaagtgataacccgggatctaaataaatccgttt

SEQ ID NO: 28 - DNA, 18 bp, Artificial Sequence
  Primer
cctagctaaatttaatgt

SEQ ID NO: 29 - DNA, 18 bp, Artificial Sequence
  Primer
cctagctaaatttaatga

SEQ ID NO: 30 - DNA, 21 bp, Artificial Sequence
  Primer
taattttccccaagtagcagg

SEQ ID NO: 31 - DNA, 1614 bp, Artificial Sequence
  recombinant nucleotide encoding four heterologous antigen sequences operably linked to a tag sequence
atgcatggagatacacctacattgcatgaatatatgttagatttgcaaccagagacaactgatctctactgttatgaacaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaatgcatcaaaaacgtacagcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttatatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgatgcatggacctaaggcaacattgcaagacattgtattgcatttagagccccaaaatgaaattccggttgaccttctatgtcacgaacaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgtcataaatgtatcgatttttattcaagaattagagaattaagacattattcagactctgtgtatggagacacattggaaaaactaactaacactgggttatacaatttattaataaggtgcctgcggtgtcagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtagcacgttctattattaacttcgaaaaattatctcatcatcatcatcatcattaataa

SEQ ID NO: 32 - DNA, 2943 bp, Artificial Sequence
  recombinant polypeptide comprising four heterologous antigen sequences operably linked at the N-terminus to truncated LLO and a peptide tag sequence at the C-terminus
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagatgcatggagatacacctacattgcatgaatatatgttagatttgcaaccagagacaactgatctctactgttatgaacaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaatgcatcaaaaacgtacagcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttatatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgatgcatggacctaaggcaacattgcaagacattgtattgcatttagagccccaaaatgaaattccggttgaccttctatgtcacgaacaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgtcataaatgtatcgatttttattcaagaattagagaattaagacattattcagactctgtgtatggagacacattggaaaaactaactaacactgggttatacaatttattaataaggtgcctgcggtgtcagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtagcacgttctattattaacttcgaaaaattatctcatcatcatcatcatcattaataa

SEQ ID NO: 33 - protein, 979 aa, Artificial Sequence
  recombinant polypeptide tLLO-HPV16E7-HPV16E6- HPV18E7-HPV16E6 SIINFEKL-S-6xHIS tag
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLMHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQVARSIINFEKLSHHHHHH

SEQ ID NO: 34 - DNA, 6523 bp, Artificial Sequence
  plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 35 - DNA, 36 bp, Artificial Sequence
  Primer
cggaattcggatccgcgccaaatcattggttgattg

SEQ ID NO: 36 - DNA, 37 bp, Artificial Sequence
  Primer
gcgagtcgacgtcggggttaatcgtaatgcaattggc

SEQ ID NO: 37 - DNA, 35 bp, Artificial Sequence
  Primer
gcgagtcgacccatacgacgttaattcttgcaatg

SEQ ID NO: 38 - DNA, 39 bp, Artificial Sequence
  Primer
gatactgcagggatccttcccttctcggtaatcagtcac

SEQ ID NO: 39 - DNA, 19 bp, Artificial Sequence
  Primer
tgggatggccaagaaattc

SEQ ID NO: 40 - DNA, 22 bp, Artificial Sequence
  Primer
ctaccatgtcttccgttgcttg

SEQ ID NO: 41 - DNA, 364 bp, Artificial Sequence
  Lovaxin_C_pGG55
ccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcgtgattatctcaaaagatatgcccctaaattagatgaatggttttatttagcatgtcctgctacttggggaaaattaaattaa

SEQ ID NO: 42 - DNA, 364 bp, Listeria monocytogenes
ccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa

(19) United States
(12) Patent Application Publication
(10) Pub. No.: US 2018/0280487 A1
(43) Pub. Date: Oct. 4, 2018

(54) RECOMBINANT LISTERIA VACCINE STRAINS AND METHODS OF USING THE SAME IN CANCER IMMUNOTHERAPY

(71) Applicant: ADVAXIS, INC., Princeton, NJ (US)

(72) Inventors: Robert Petit, Newtown, PA (US); Kyle Perry, Grand Blanc, MI (US)

(21) Appl. No.: 15/767,625

(22) PCT Filed: Oct. 14, 2016
(86) PCT No.: PCT/US16/57220

§ 371 Date: Apr. 11, 2018

Related U.S. Application Data
(60) Provisional application No. 62/241,712, filed on Oct. 14, 2015.

(51) Int. Cl. A61K 39/00; A61K 39/02; A61K 39/39; A61K 39/245; A61P 35/00; A61K 35/74

(52) U.S. Cl. CPC A61K 39/0011; A61K 39/02; A61K 39/39; A61K 2039/545; A61P 35/00; A61K 35/74; A61K 2039/572; A61K 39/245

(57) ABSTRACT

The present invention provides methods of treating, protecting against, and inducing an immune response against a human papillomavirus-associated tumor or cancer, comprising the step of administering to a subject a recombinant Listeria strain expressing a construct comprising at least one human papillomavirus antigen.

### FIELD OF INVENTION

The present invention provides methods of treating, protecting against, and inducing an immune response against a human papillomavirus-associated tumor or cancer, comprising the step of administering to a subject a recombinant Listeria strain expressing a construct comprising at least one human papillomavirus antigen.

### BACKGROUND OF THE INVENTION

The prevalence of human papillomavirus (HPV)-associated oropharyngeal cancer (HPVOPC) is increasing in the USA (225% from 1988 to 2004). HPVOPC patients tend to be younger and have a favorable prognosis, with a 69% reduction in the risk of death compared with HPV-negative patients. However most HPVOPC patients present with advanced stage, and standard chemoradiation regimens can be associated with significant toxicity. Thus the patients who have a good prognosis are paradoxically at greater risk of therapy-related long-term poor quality-of-life outcomes. Immunotherapy has the potential to reduce toxicity through de-escalation of chemoradiation regimens, and potentially enhance long-term disease control.

The HR-HPV E6 and E7 proteins are consistently expressed in dysplasias and carcinomas, disrupting the cell cycle regulatory proteins p53 and pRb, respectively. The obligatory expression of E6 and E7 by both dysplastic and invasive malignant lesions, as well as the viral origin of these proteins, make them excellent targets for HPV therapeutic vaccines.

Listeria monocytogenes (Lm) is a food-borne gram-positive bacterium that can occasionally cause disease in humans, in particular elderly individuals, newborns, pregnant women and immunocompromised individuals. In addition to strongly activating innate immunity and inducing a cytokine response that enhances antigen-presenting cell (APC) function, Lm has the ability to replicate in the cytosol of APCs after escaping from the phagolysosome, mainly through the action of the listeriolysin O (LLO) protein. This unique intracellular life cycle allows antigens secreted by Lm to be processed and presented in the context of both MHC class I and II molecules, resulting in potent cytotoxic CD8⁺ and Th1 CD4⁺ T-cell-mediated immune responses.

The present invention addresses the need for improved treatment modalities in patients having human papillomavirus (HPV)-associated cancer by providing a Listeria monocytogenes-based immunotherapy comprising several HPV antigens and use of the same for preventing and treating HPV-related cancers.

### SUMMARY OF THE INVENTION

In one aspect, the present invention relates to an immunogenic composition comprising a recombinant Listeria strain comprising a recombinant nucleic acid, said nucleic acid comprising a first open reading frame encoding a recombinant polypeptide comprising an N-terminal fragment of an LLO protein operably linked to or fused to at least one heterologous antigen or fragment thereof, wherein said recombinant nucleic acid further comprises a second open reading frame encoding a metabolic enzyme. In another aspect, said recombinant Listeria comprises a mutation, deletion or inactivation of an endogenous dal, dat, and actA gene.

In yet further aspect, the present invention relates to an immunogenic composition comprising a recombinant Listeria strain comprising a recombinant nucleic acid, said nucleic acid comprising a first open reading frame encoding a recombinant polypeptide comprising an N-terminal fragment of an LLO protein operably linked or fused to four heterologous antigens or fragments thereof, wherein said recombinant nucleic acid further comprises a second open reading frame encoding a metabolic enzyme, and wherein said four heterologous antigens are selected from the group consisting of HPV16 E6, HPV16 E7, HPV18 E6, and HPV18 E7 antigens. In another aspect, said recombinant Listeria comprises a mutation, deletion or inactivation of an endogenous dal, dat, and actA gene.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Lm-E7 and Lm-LLO-E7 use different expression systems to express and secrete E7. Lm-E7 was generated by introducing a gene cassette into the orfZ domain of the L. monocytogenes genome (A). The hly promoter drives expression of the hly signal sequence and the first five amino acids (AA) of LLO followed by HPV-16 E7. B), Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)-(no-TC-1 control).

FIG. 5. A. Induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive). B. Induction and penetration of E7 specific CD8⁺ cells in the spleens and tumors of the mice described for (A).

FIG. 6. Listeria constructs containing PEST regions induce a higher percentage of E7-specific lymphocytes within the tumor. A. representative data from 1 experiment. B. average and SE of data from all 3 experiments.

FIG. 7A. Effect of passaging on bacterial load (virulence) of recombinant Listeria vaccine vectors. Top panel. Lm-Gag. Bottom panel. Lm-LLO-E7. FIG. 7B. Effect of passaging on bacterial load of recombinant Lm-E7 in the spleen. Average CFU of live bacteria per milliliter of spleen homogenate from four mice is depicted.

FIG. 8 shows induction of antigen-specific CD8⁺ T-cells for HIV-Gag and LLO after administration of passaged Lm-Gag versus unpassaged Lm-Gag. Mice were immunized with 10³ (A, B, E, F) or 10⁵ (C, D, G, H) CFU passaged Listeria vaccine vectors, and antigen-specific T-cells were analyzed. B, D, F, H: unpassaged Listeria vaccine vectors. A-D immune response to MHC class I HIV-Gag peptide. E-H: immune response to an LLO peptide. I: splenocytes from mice immunized with 10⁵ CFU passaged Lm-Gag stimulated with a control peptide from HPV E7.

FIG. 9A shows plasmid isolation throughout LB stability study. FIG. 9B shows plasmid isolation throughout TB stability study. FIG. 9C shows quantitation of TB stability study.

FIG. 10 shows numbers of viable bacteria chloramphenicol (CAP)-resistant and CAP-sensitive colony-forming units (CFU) from bacteria grown in LB. Dark bars: CAP⁺, white bars: CAP⁻. The two dark bars and two white bars for each time point represent duplicate samples.

FIG. 11 shows numbers of viable bacteria CAP-resistant and CAP-sensitive CFU from bacteria grown in TB. Dark bars: CAP⁺, white bars: CAP. The two dark bars and two white bars for each time point represent duplicate samples.

FIG. 12. Actual chromatograms showing the region of the D133V mutation (arrows). The mixture ratio is shown in parentheses.

FIG. 13. Representation of the location of the ADV451, 452 and 453 primers and the segment of the prfA gene amplified in the reaction.

FIG. 14. Specificity of the PCR reaction using primers ADV451 and ADV453.

FIG. 15. Specificity of the PCR reaction using primers ADV452 and ADV453.

FIG. 16. Sensitivity of the PCR reaction to detect the wild-type prfA sequence using the primer ADV452 and 1 ng as the initial amount of DNA.

FIG. 17. Sensitivity of the PCR reaction to detect the wild-type prfA sequence using the primer ADV452 and 5 ng as the initial amount of DNA.

FIG. 18. Average density of the bands from the PCR depicted in FIG. 16.

FIG. 19. Average density of the bands from the PCR depicted in FIG. 17.

FIG. 20. Validation of the PCR reaction to detect the wild-type prfA sequence using the primer ADV452.

FIG. 21. Average density of the bands from the PCR depicted in FIG. 16.

FIG. 22. Analysis of the D133V PrfA mutation in the Lm-LLO-E7. A, Original image used for densitometry; B, Image was digitally enhanced to facilitate the visualization of the low density bands.

FIG. 23. (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 714bp corresponding to the klk3 gene, lacking the secretion signal sequence of the wild type protein.

FIG. 24. (A) Map of the pADV134 plasmid. (B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (C) Map of the pADV142 plasmid. (D) Western blot showed the expression of LLO-PSA fusion protein using anti-PSA and anti-LLO antibody.

FIG. 25. Tumor volume measured in mice inoculated with TC1 tumors that express HPV 16 E6&E7; that were treated with (A) decreasing doses Lmdda-HPVQuad or LmddA274, (B) 1×10⁸ Lmdda-HPVQuad, 1×10⁷ Lmdda-HPVQuad, or LmddA274, (C) 1×10⁸ Lmdda-HPVQuad, 2.5×10⁷ Lmdda-HPVQuad, or LmddA274, (D) 1×10⁸ Lmdda-HPVQuad, 5×10⁷ Lmdda-HPVQuad, or LmddA274.

FIG. 26. (A) Tumor volume measured in mice inoculated with TC1 tumors that express HPV 16 E6&E7; that were treated with decreasing doses AXAL/Lm-E7 or with an empty vector Lm-tLLO. (B) Tumor volume measured in mice inoculated with TC1 tumors that express HPV 16 E6&E7; that were treated with decreasing doses Lmdda-HPVQuad or with an empty vector Lm-tLLO. N=5-10 animals per group.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The present invention provides compositions comprising a recombinant Listeria strain expressing a recombinant polypeptide comprising a listeriolysin O (LLO) fragment and at least one antigen or a fragment associated with a disease thereof and methods of treating, protecting against, and inducing an immune response against a disease, comprising the step of administering the composition comprising the recombinant Listeria strain.

In one embodiment, the nucleic acid molecule disclosed herein comprises a first open reading frame encoding recombinant polypeptide comprising a heterologous antigen or fragment thereof. In another embodiment, the recombinant polypeptide further comprises an N-terminal LLO fused to the heterologous antigen. In another embodiment, the nucleic acid molecule disclosed herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains disclosed herein comprise a mutation, a deletion or inactivation in the genomic dal, dat, or actA genes. In another embodiment, the Listeria strains disclosed herein comprise a mutation, a deletion or inactivation in the genomic dal, dat, and actA genes. In another embodiment, the Listeria lack the genomic dal, dat or actA genes. In another embodiment, the Listeria lack the genomic dal, dat and actA genes.

In one embodiment, disclosed herein is a nucleic acid construct encoding a recombinant polypeptide comprising the following elements: a N-terminal truncated LLO (tLLO) fused to a first heterologous antigen amino acid acid sequence, wherein said first heterologous antigen AA sequence is operatively linked to a second heterologous antigen AA sequence, wherein said second heterologous antigen AA sequence is operatively linked to at least one additional heterologous antigen amino acid sequence. In another embodiment, said elements are arranged or are operatively linked from N-terminus to C-terminus. In another embodiment, the nucleic acid sequence comprises one or more linker sequences incorporated between at least one first heterologous antigen and at least one second heterologous antigen. In another embodiment, the nucleic acid sequence comprises at least two different linker sequences incorporated between at least one first heterologous antigen and at least one second heterologous antigen to at least one third heterologous antigen. In another embodiment, one or more linker(s) is a 4xglycine linker

In another embodiment, a recombinant Listeria strain disclosed herein comprises a recombinant nucleic acid construct comprising a first open reading frame encoding a recombinant polypeptide comprising an N-terminal fragment of an LLO protein operably linked to or fused to at least one heterologous antigen or fragment thereof. In another embodiment, the recombinant Listeria strain comprises a recombinant nucleic acid construct comprising a first open reading frame encoding a recombinant polypeptide comprising an N-terminal fragment of an LLO protein operably linked or fused to more than one heterologous antigen or fragment thereof. In another embodiment, the recombinant Listeria strain comprises a recombinant nucleic acid construct comprising a first open reading frame encoding a recombinant polypeptide comprising an N-terminal fragment of an LLO protein operably linked or fused to two heterologous antigens or functional fragments thereof. In another embodiment, the recombinant Listeria strain comprises a recombinant nucleic acid construct comprising a first open reading frame encoding a recombinant polypeptide comprising an N-terminal fragment of an LLO protein operably linked or fused to two to four heterologous antigens or functional fragments thereof. In another embodiment, the recombinant Listeria strain comprises a recombinant nucleic acid construct comprising a first open reading frame encoding a recombinant polypeptide comprising an N-terminal fragment of an LLO protein operably linked or fused to four heterologous antigens or functional fragments thereof. In one embodiment, two to four heterologous antigens are operably linked in tandem and fused to an N-terminal LLO protein fragment at the N-terminus of the most N-terminus heterologous antigen. In another embodiment, the recombinant Listeria strain comprises a recombinant nucleic acid construct comprising a first open reading frame encoding a recombinant polypeptide comprising an N-terminal fragment of an LLO protein operably linked or fused to more than four heterologous antigens or fragments thereof.

The N-terminal LLO protein fragment and heterologous antigen are, in another embodiment, fused directly to one another. In another embodiment, the genes encoding the N-terminal LLO protein fragment and the heterologous antigen are fused directly to one another. In another embodiment, the N-terminal LLO protein fragment and the heterologous antigen are attached via a linker peptide. In another embodiment, the N-terminal LLO protein fragment and the heterologous antigen are attached via a heterologous peptide. In another embodiment, the N-terminal LLO protein fragment is N-terminal to the heterologous antigen. In another embodiment, the N-terminal LLO protein fragment is the N-terminal-most portion of the fusion protein.

In one embodiment, the recombinant Listeria strain comprises a quadravalent episomal expression vector, the vector comprising a first, second, third, and fourth nucleic acid molecule, each encoding a heterologous antigenic polypeptide or a functional fragment thereof, wherein the first through fourth nucleic acid molecules each encode the heterologous antigenic polypeptide or functional fragment thereof in an open reading frame with an N-terminal or truncated or detoxified Listeriolysin O protein (LLO). In another embodiment, the PEST-containing polypeptide is an N-terminal or a truncated ActA protein. In another embodiment, the PEST-containing peptide is a PEST amino acid sequence.

In one embodiment, the heterologous antigens expressed by quadravalent expression vector are HPV16 E6 and E7, and HPV18 E6 and E7.

In one embodiment, the present invention provides a method of inducing an anti-tumor or an anti-cancer immune response in a human subject, the method comprising the step of administering to said subject a composition comprising a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, wherein said recombinant nucleic acid further comprises a second open reading frame encoding a mutant prfA gene or a metabolic enzyme, thereby inducing an immune response against a tumor or a cancer. In another embodiment, said immune response reduces the need for said subject to receive chemotherapy or radiation. In another embodiment, said immune response eliminates the need for said subject to receive chemotherapy or radiation. In another embodiment, said immune response reduces the severity of side effects associated with administration of a chemotherapy or radiation to said subject by allowing the patient to be treated with lower doses of chemotherapy or radiation.

In one embodiment, the present invention provides a method of inducing an anti-tumor or an anti-cancer immune response in a human subject, the method comprising the step of administering to said subject a composition comprising a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, wherein said recombinant nucleic acid further comprises a second open reading frame encoding a metabolic enzyme, wherein said immune response reduces the need for said subject to receive chemotherapeutic or radiation treatment, thereby inducing an immune response against a tumor or a cancer. In another embodiment, said Listeria comprises a mutation in the endogenous dal/dat and actA genes.

It will be appreciated by a skilled artisan that the composition comprising a recombinant Listeria provided herein may be administered in combination with other treatment modalities, including, but not limited to, chemotherapy, radiation, and/or checkpoint inhibitors. In another embodiment, administration of the Listeria disclosed herein or the Listeria-based immunotherapy disclosed herein is able to reduce the need of a subject having a tumor or a cancer to receive chemotherapeutic or radiation treatment. In another embodiment, administration of the Listeria disclosed herein or the Listeria-based immunotherapy disclosed herein is able to eliminate the need for a subject having a tumor or cancer to receive radiation or chemotherapy. In another embodiment, administration of the Listeria disclosed herein or the Listeria-based immunotherapy disclosed herein is able to reduce the severity of side effects associated with a radiation or chemotherapy treatment in a subject having a tumor or cancer.

In one embodiment, the present invention also provides methods for inducing an anti-disease cytotoxic T-cell (CTL) response in a human subject and treating disorders, and symptoms associated with said disease comprising administration of the recombinant Listeriastrain.

In one embodiment, disclosed herein is a recombinant Listeria strain, said recombinant Listeria strain comprising a recombinant nucleic acid, said nucleic acid comprising a first open reading frame encoding a recombinant polypeptide comprising a first an N-terminal fragment of an LLO protein fused to a heterologous antigen or fragment thereof, and wherein said recombinant nucleic acid further comprises a second open reading frame encoding a mutant prfA gene. In one embodiment, the mutant prfA gene is one that encodes a point mutation from amino acid D (which also known as “Asp,” “Aspartate”or “Aspartic acid”) to amino acid V (which is also known as “Val,” or “Valine”) at amino acid position 133. In one embodiment, a recombinant Listeria strain disclosed herein comprises a mutation or deletion in the endogenous prfA gene. In another embodiment, a chromosomal mutation or deletion in a prfA gene in a Listeria disclosed herein is complemented via a plasmid comprising a nucleic acid sequence encoding a mutant prfA gene encoding a mutant PrfA protein comprising a D133V amino acid substitution. In another embodiment, a mutant PrfA protein comprising a D133V amino acid substitution complements an endogenous prfA mutation in a Listeria disclosed herein.

In another embodiment, the recombinant Listeria is an attenuated Listeria. It will be appreciated that the terms “attenuation” or “attenuated” may encompass a bacterium, virus, parasite, infectious organism, prion, tumor cell, gene in the infectious organism, and the like, that is modified to reduce toxicity to a host. The host can be a human or animal, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. In one embodiment, attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results in an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold. In another embodiment, attenuation results in an increase in the LD₅₀ and/or an increase in the rate of clearance by at least 25%. In another embodiment, attenuation results in an increase in the LD₅₀ and/or an increase in the rate of clearance by 3-5 fold. In other embodiments, attenuation results in an increase in the LD₅₀ and/or an increase in the rate of clearance by 5-10 fold, 11-20 fold, 21-30 fold, 31-40 fold, 41-50 fold, 51-100 fold, 101-500 fold, 501-1,000 fold, 1001-10,000 fold, or 10,001-100,000 fold.

It will be well appreciated by a skilled artisan that the term “Attenuated gene” may encompass a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, inversions, truncations, and frameshift mutations in regulatory regions of the gene, coding regions of the gene, non-coding regions of the gene, or any combination thereof.

In one embodiment, disclosed herein is a method for inducing an immune response against a tumor or a cancer in a human subject, the method comprising the step of administering to said subject a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, is, wherein said recombinant nucleic acid further comprises a second open reading frame encoding a mutant PrfA protein, thereby inducing an immune response against a tumor or a cancer In one embodiment, the present invention provides a method of treating a cancer in a human subject, comprising the step of administering to the subject the recombinant Listeria strain disclosed herein. In another embodiment, the present invention provides a method of protecting a human subject against a cervical cancer, comprising the step of administering to the subject the recombinant Listeria strain disclosed herein. In another embodiment, the recombinant Listeria strain expresses the recombinant polypeptide. In another embodiment, the recombinant Listeria strain comprises a plasmid that encodes the recombinant polypeptide. In another embodiment, the method further comprises the step of boosting the human subject with a recombinant Listeria strain of the present invention. In another embodiment, the method further comprises the step of boosting the human subject with an immunogenic composition comprising a heterologous antigen or fragment thereof disclosed herein. In another embodiment, the method further comprises the step of boosting the human subject with an immunogenic composition that directs a cell of the subject to express the heterologous antigen. In another embodiment, the cell is a tumor cell. In another embodiment, the method further comprises the step of boosting the human subject with the vaccine of the present invention.

In one embodiment, the fragment thereof in the context of LLO proteins and ActA proteins disclosed herein refer to a peptide or polypeptide comprising an amino acid sequence of at least 5 contiguous amino acid residues of the LLO or ActA proteins. In another embodiment, the term refers to a peptide or polypeptide comprising an amino acid sequence of at least of at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, at least 250 contiguous amino acid residues of the amino acid sequence, at least 300 contiguous amino acid residues, at least 350 contiguous amino acid residues of, at least 400 contiguous amino acid residues, or at least 450 contiguous amino acid residues of an LLO or ActA protein or polypeptide.

In another embodiment, the fragment is a functional fragment that works as intended by the present invention (e.g. to elicit an immune response against a disease-associated antigen when in the form of an N-terminal LLO/heterologous antigen fusion protein or N-terminal ActA/heterologous antigen fusion protein). In another embodiment, the fragment is functional in a non-fused form. In another embodiment, the fragment is an immunogenic fragment.

The present invention, in certain embodiments, provides codon optimization of a nucleic acid heterologous to Listeria, or of a nucleic acid endogenous to Listeria. The optimal codons utilized by L. monocytogenes for each amino acid are shown US Patent Publication 2007/0207170, which is hereby incorporated by reference herein. A nucleic acid is codon-optimized if at least one codon in the nucleic acid is replaced with a codon that is more frequently used by L. monocytogenes for that amino acid than the codon in the original sequence.

As disclosed herein, recombinant Listeria strains expressing LLO-antigen fusions induce anti-tumor immunity (Example 1), elicit antigen-specific T cell proliferation (Example 2), generate antigen-specific, and tumor-infiltrating T cells (Example 3).

In another embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an LLO protein and at least one HPV antigen, whereby the recombinant Listeria strain induces an immune response against the HPV antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an LLO protein and at least one HPV antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of treating an HPV-related cancer. In another embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an LLO protein and at least one HPV antigen, whereby the recombinant Listeria strain induces an immune response against the heterologous antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an ActA protein and at least one HPV antigen, whereby the recombinant Listeria strain induces an immune response against the heterologous antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an ActA protein and at least one heterologous antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide. In one embodiment, the present invention provides a method of treating an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an ActA protein and at least one heterologous antigen, thereby treating an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising a PEST sequence and at least one HPV antigen, whereby the recombinant Listeria strain induces an immune response against the heterologous antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising a PEST sequence and at least one heterologous antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide. In one embodiment, the present invention provides a method of treating an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising a PEST sequence and at least one heterologous antigen, thereby treating an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

The N-terminal LLO protein fragment and at least one heterologous antigen are, in another embodiment, fused directly to one another. In another embodiment, the genes encoding the N-terminal LLO protein fragment and at least heterologous antigen are fused directly to one another. In another embodiment, the N-terminal LLO protein fragment and at least heterologous antigen are attached via a linker peptide. In another embodiment, the N-terminal LLO protein fragment and at least heterologous antigen are attached via a heterologous peptide. In another embodiment, the N-terminal LLO protein fragment is N-terminal to at least heterologous antigen. In another embodiment, the N-terminal LLO protein fragment is N-terminal to all of the heterologous antigens. In another embodiment, the N-terminal LLO protein fragment is the N-terminal-most portion of the fusion protein.

The N-terminal ActA protein fragment and at least one heterologous antigen are, in another embodiment, fused directly to one another. In another embodiment, the genes encoding the N-terminal ActA protein fragment and at least one heterologous antigen are fused directly to one another. In another embodiment, the N-terminal ActA protein fragment and at least one heterologous antigen are attached via a linker peptide. In another embodiment, the N-terminal ActA protein fragment and at least one heterologous antigen are attached via a heterologous peptide. In another embodiment, the N-terminal ActA protein fragment is N-terminal to at least heterologous antigen. In another embodiment, the N-terminal ActA protein fragment is N-terminal to all of the heterologous antigens. In another embodiment, the N-terminal ActA protein fragment is the N-terminal-most portion of the fusion protein.

The PEST sequence and at least one heterologous antigen are, in another embodiment, fused directly to one another. In another embodiment, the genes encoding the

PEST sequence and at least one heterologous antigen are fused directly to one another. In another embodiment, the PEST sequence and at least one heterologous antigen are attached via a linker peptide. In another embodiment, the PEST sequence and at least one heterologous antigen are attached via a heterologous peptide. In another embodiment, the PEST sequence is N-terminal to at least heterologous antigen. In another embodiment, the PEST sequence is N-terminal to all of the heterologous antigens. In another embodiment, the PEST sequence is the N-terminal-most portion of the fusion protein.

In another embodiment, the present invention provides a method for vaccinating a human subject against an HPV, comprising the step of administering to the subject the recombinant Listeria strain disclosed herein, wherein the Listeria expresses an HPV E7 antigen and wherein the Listeria expresses a mutant PrfA protein. In another embodiment, the mutant prfA gene encodes a D133V mutation in PrfA protein. In another embodiment, the mutant prfA gene is in a plasmid in said recombinant Listeria. In another embodiment, the recombinant Listeria strain expresses the recombinant polypeptide. In another embodiment, the recombinant Listeria strain comprises a plasmid that encodes the recombinant polypeptide.

In one embodiment, at least one of two to four heterologous antigens or functional fragments thereof disclosed herein is expressed from an extrachromosomal plasmid in said Listeria and at least one of two to four heterologous antigens or functional fragments thereof disclosed herein is expressed from the genome of said Listeria.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region. In another embodiment the term “operably linked” as used herein means that several open reading frames are fused in a way that forms a single continuous reading frame resulting in expression of a protein that incorporates sequences of the original proteins arranged in succession.

In one embodiment, “fused” refers to operable linkage by covalent bonding. In one embodiment, the term includes recombinant fusion (of nucleic acid sequences or open reading frames thereof). In another embodiment, the term includes chemical conjugation.

In one embodiment, the recombinant nucleic acid comprising a first open reading frame encoding a recombinant polypeptide comprising an N-terminal fragment of an LLO protein operably linked or fused to multiple heterologous antigens or fragments thereof is further operably linked to a tag sequence. In one embodiment the tag is positioned on the amino terminal of the first open reading frame. In another embodiment the tag is positioned on the carboxy terminal of the first open reading frame and is operatively linked to the last heterologous antigen sequence. In another embodiment, each nucleic acid construct comprises at least 1 stop codon following the tag sequence. In another embodiment, each nucleic acid construct comprises 2 stop codons following the tag sequence

In one embodiment the tag sequence comprises the tag sequence comprises a C-terminal SIINFEKL and 6 His amino acids. In another embodiment, the tag sequence is an amino acid or nucleic acid sequence that allows for easy detection of the fusion polypeptide. In another embodiment, the tag sequence is an amino acid or nucleic acid sequence that is useful for confirmation of secretion of a fusion polypeptide disclosed herein. It will be appreciated by a skilled artisan that the sequences for the tags may be incorporated into the fusion peptide sequences on the plasmid or phage vector. These tags may be expressed and the antigenic epitopes presented allow a clinician to follow the immunogenicity of the secreted peptide by following immune responses to these “tag” sequence peptides. Such immune response can be monitored using a number of reagents including but not limited to, monoclonal antibodies and DNA or RNA probes specific for these tags.

In one embodiment, a recombinant polypeptide disclosed herein is expressed and secreted by a recombinant Listeria disclosed herein. In another embodiment, secretion of the antigen, or polypeptides (fusion or chimeric) disclosed herein is detected using a protein, molecule or antibody (or fragment thereof) that specifically binds to a polyhistidine (His) tag. In another embodiment, the fusion polypeptide disclosed herein is expressed and secreted by a recombinant Listeria disclosed herein. In another embodiment, secretion of the antigen, or recombinant polypeptide disclosed herein is detected using an antibody, protein or molecule that binds a SIINFEKL-S-6xHIS tag. In another embodiment, the recombinant polypeptide disclosed herein comprise any other tag know in the art, including, but not limited to chitin binding protein (CBP), maltose binding protein (MBP), and glutathione-S-transferase (GST), thioredoxin (TRX) and poly(NANP).

In another embodiment, the four operably linked heterologous antigens encoded by the first open reading frame of the recombinant nucleic acid comprise HPV16 E6 and E7, and HPV18 E6 and E7. In another embodiment the recombinant polypeptide expressed from a first open reading frame disclosed herein and secreted by a recombinant Listeria disclosed herein comprises the following structure: N-terminal LLO-HPV16 E7-HPV16 E6-HPV18 E7-HPV18 E6-SIINFEKL-S-6xHIS tag. In another embodiment, the recombinant polypeptide disclosed herein comprises SEQ ID NO: 33. In another embodiment, the recombinant polypeptide disclosed herein is encoded by SEQ ID NO: 32. In another embodiment, the four operably linked heterologous antigens are arranged in any other order. In another embodiment, HPV E6 sequences (SEQ ID No: 15 and SEQ ID No: 16) and HPV E7 sequences (SEQ ID No: 13 and SEQ ID No: 14) are operably linked thereby encoding a quadravalent heterologous antigen sequence. In another embodiment, the quadravalent antigen sequence is further operably linked to truncated LLO at the N-terminus or at the C-terminus. In another embodiment the quadravalent antigen sequence is operably linked to truncated LLO and a peptide tag sequence.

In one embodiment, a recombinant polypeptide disclosed herein comprises the following structure from N- to C-terminus: antigen 1-antigen 2-antigen 3-antigen 4. In one embodiment, a recombinant polypeptide disclosed herein comprises the following structure from N- to C-terminus: N-terminal LLO-antigen 1-antigen 2-antigen 3-antigen 4. In one embodiment, a recombinant polypeptide disclosed herein comprises the following structure from N- to C-terminus: N-terminal LLO-antigen 1-antigen 2-antigen 3-antigen 4-SIINFEKL-S-6xHIS tag. In one embodiment, a recombinant polypeptide disclosed herein comprises the following structure from N- to C-terminus: N-terminal LLO-HPV16 E7-HPV16 E6-HPV18 E7-HPV18 E6-SIINFEKL-S-6xHIS tag. In one embodiment, a recombinant polypeptide disclosed herein is encoded by a nucleic acid sequence comprising SEQ ID NO: 31. In one embodiment, the recombinant nucleotide encoding four heterologous antigen sequences operably linked to a tag sequence comprises the sequence set forth in SEQ ID NO: 31:

atgcatggagatacacctacattgcatgaatatatgttagatttgcaacc
agagacaactgatctctactgttatgaacaattaaatgacagctcagagg
aggaggatgaaatagatggtccagctggacaagcagaaccggacagagcc
cattacaatattgtaaccttttgttgcaagtgtgactctacgcttcggtt
gtgcgtacaaagcacacacgtagacattcgtactttggaagacctgttaa
tgggcacactaggaattgtgtgccccatctgttctcagaaaccaatgcat
caaaaacgtacagcaatgtttcaggacccacaggagcgacccagaaagtt
accacagttatgcacagagctgcaaacaactatacatgatataatattag
aatgtgtgtactgcaagcaacagttactgcgacgtgaggtatatgacttt
gcttttcgggatttatgcatagtatatagagatgggaatccatatgctgt
atgtgataaatgtttaaagttttattctaaaattagtgagtatagacatt
attgttatagtttatatggaacaacattagaacagcaatacaacaaaccg
ttgtgtgatttgttaattaggtgtattaactgtcaaaagccactgtgtcc
tgaagaaaagcaaagacatctggacaaaaagcaaagattccataatataa
ggggtcggtggaccggtcgatgtatgtcttgttgcagatcatcaagaaca
cgtagagaaacccagctgatgcatggacctaaggcaacattgcaagacat
tgtattgcatttagagccccaaaatgaaattccggttgaccttctatgtc
acgaacaattaagcgactcagaggaagaaaacgatgaaatagatggagtt
aatcatcaacatttaccagcccgacgagccgaaccacaacgtcacacaat
gttgtgtatgtgttgtaagtgtgaagccagaattgagctagtagtagaaa
gctcagcagacgaccttcgagcattccagcagctgtttctgaacaccctg
tcctttgtgtgtccgtggtgtgcatcccagcagatggcgcgctttgagga
tccaacacggcgaccctacaagctacctgatctgtgcacggaactgaaca
cttcactgcaagacatagaaataacctgtgtatattgcaagacagtattg
gaacttacagaggtatttgaatttgcatttaaagatttatttgtggtgta
tagagacagtataccgcatgctgcatgtcataaatgtatcgatttttatt
caagaattagagaattaagacattattcagactctgtgtatggagacaca
ttggaaaaactaactaacactgggttatacaatttattaataaggtgcct
gcggtgtcagaaaccgttgaatccagcagaaaaacttagacaccttaatg
aaaaacgacgatttcacaacatagctgggcactatagaggccagtgccat
tcgtgctgcaaccgagcacgacaggaacgactccaacgacgcagagaaac
acaagtagcacgttctattattaacttcgaaaaattatctcatcatcatc
atcatcattaataa

In one embodiment, a nucleic acid construct encoding a recombinant polypeptide comprising four heterologous antigen sequences operably linked at the N-terminus to truncated LLO and a peptide tag sequence at the C-terminus comprises SEQ ID NO: 32:

atgaaaaaaataatgctagtttttattacacttatattagttagtctacc
aattgcgcaacaaactgaagcaaaggatgcatctgcattcaataaagaaa
attcaatttcatccatggcaccaccagcatctccgcctgcaagtcctaag
acgccaatcgaaaagaaacacgcggatgaaatcgataagtatatacaagg
attggattacaataaaaacaatgtattagtataccacggagatgcagtga
caaatgtgccgccaagaaaaggttacaaagatggaaatgaatatattgtt
gtggagaaaaagaagaaatccatcaatcaaaataatgcagacattcaagt
tgtgaatgcaatttcgagcctaacctatccaggtgctctcgtaaaagcga
attcggaattagtagaaaatcaaccagatgttctccctgtaaaacgtgat
tcattaacactcagcattgatttgccaggtatgactaatcaagacaataa
aatagttgtaaaaaatgccactaaatcaaacgttaacaacgcagtaaata
cattagtggaaagatggaatgaaaaatatgctcaagcttatccaaatgta
agtgcaaaaattgattatgatgacgaaatggcttacagtgaatcacaatt
aattgcgaaatttggtacagcatttaaagctgtaaataatagcttgaatg
taaacttcggcgcaatcagtgaagggaaaatgcaagaagaagtcattagt
tttaaacaaatttactataacgtgaatgttaatgaacctacaagaccttc
cagatttttcggcaaagctgttactaaagagcagttgcaagcgcttggag
tgaatgcagaaaatcctcctgcatatatctcaagtgtggcgtatggccgt
caagtttatttgaaattatcaactaattcccatagtactaaagtaaaagc
tgcttttgatgctgccgtaagcggaaaatctgtctcaggtgatgtagaac
taacaaatatcatcaaaaattcttccttcaaagccgtaatttacggaggt
tccgcaaaagatgaagttcaaatcatcgacggcaacctcggagacttacg
cgatattttgaaaaaaggcgctacttttaatcgagaaacaccaggagttc
ccattgcttatacaacaaacttcctaaaagacaatgaattagctgttatt
aaaaacaactcagaatatattgaaacaacttcaaaagcttatacagatgg
aaaaattaacatcgatcactctggaggatacgttgctcaattcaacattt
cttgggatgaagtaaattatgatctcgagatgcatggagatacacctaca
ttgcatgaatatatgttagatttgcaaccagagacaactgatctctactg
ttatgaacaattaaatgacagctcagaggaggaggatgaaatagatggtc
cagctggacaagcagaaccggacagagcccattacaatattgtaaccttt
tgttgcaagtgtgactctacgcttcggttgtgcgtacaaagcacacacgt
agacattcgtactttggaagacctgttaatgggcacactaggaattgtgt
gccccatctgttctcagaaaccaatgcatcaaaaacgtacagcaatgttt
caggacccacaggagcgacccagaaagttaccacagttatgcacagagct
gcaaacaactatacatgatataatattagaatgtgtgtactgcaagcaac
agttactgcgacgtgaggtatatgactttgcttttcgggatttatgcata
gtatatagagatgggaatccatatgctgtatgtgataaatgtttaaagtt
ttattctaaaattagtgagtatagacattattgttatagtttatatggaa
caacattagaacagcaatacaacaaaccgttgtgtgatttgttaattagg
tgtattaactgtcaaaagccactgtgtcctgaagaaaagcaaagacatct
ggacaaaaagcaaagattccataatataaggggtcggtggaccggtcgat
gtatgtcttgttgcagatcatcaagaacacgtagagaaacccagctgatg
catggacctaaggcaacattgcaagacattgtattgcatttagagcccca
aaatgaaattccggttgaccttctatgtcacgaacaattaagcgactcag
aggaagaaaacgatgaaatagatggagttaatcatcaacatttaccagcc
cgacgagccgaaccacaacgtcacacaatgttgtgtatgtgttgtaagtg
tgaagccagaattgagctagtagtagaaagctcagcagacgaccttcgag
cattccagcagctgtttctgaacaccctgtcctttgtgtgtccgtggtgt
gcatcccagcagatggcgcgctttgaggatccaacacggcgaccctacaa
gctacctgatctgtgcacggaactgaacacttcactgcaagacatagaaa
taacctgtgtatattgcaagacagtattggaacttacagaggtatttgaa
tttgcatttaaagatttatttgtggtgtatagagacagtataccgcatgc
tgcatgtcataaatgtatcgatttttattcaagaattagagaattaagac
attattcagactctgtgtatggagacacattggaaaaactaactaacact
gggttatacaatttattaataaggtgcctgcggtgtcagaaaccgttgaa
tccagcagaaaaacttagacaccttaatgaaaaacgacgatttcacaaca
tagctgggcactatagaggccagtgccattcgtgctgcaaccgagcacga
caggaacgactccaacgacgcagagaaacacaagtagcacgttctattat
taacttcgaaaaattatctcatcatcatcatcatcattaataa.

In another embodiment, the nucleic acid construct is a variant of SEQ ID NO:32. In another embodiment, the nucleic acid construct is a homolog of SEQ ID NO:32.

In another embodiment, the amino acid sequence of the recombinant polypeptide encoded by the nucleic acid construct disclosed herein tLLO-HPV16E7-HPV16E6-HPV18E7-HPV16E6 SIINFEKL-S-6xHIS tag comprises SEQ ID NO: 33

(SEQ ID NO: 33)

MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYPNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDLEMHGDTPT
LHEYMLDLQPETTDLYCYEQLNDSSEEEDEIDGPAGQAEPDRAHYNIVTF
CCKCDSTLRLCVQSTHVDIRTLEDLLMGTLGIVCPICSQKPMHQKRTAMF
QDPQERPRKLPQLCTELQTTIHDIILECVYCKQQLLRREVYDFAFRDLCI
VYRDGNPYAVCDKCLKFYSKISEYRHYCYSLYGTTLEQQYNKPLCDLLIR
CINCQKPLCPEEKQRHLDKKQRFHNIRGRWTGRCMSCCRSSRTRRETQLM
HGPKATLQDIVLHLEPQNEIPVDLLCHEQLSDSEEENDEIDGVNHQHLPA
RRAEPQRHTMLCMCCKCEARIELVVESSADDLRAFQQLFLNTLSFVCPWC
ASQQMARFEDPTRRPYKLPDLCTELNTSLQDIEITCVYCKTVLELTEVFE
FAFKDLFVVYRDSIPHAACHKCIDFYSRIRELRHYSDSVYGDTLEKLTNT
GLYNLLIRCLRCQKPLNPAEKLRHLNEKRRFHNIAGHYRGQCHSCCNRAR
QERLQRRRETQVARSIINFEKLSHHHHHH.

In another embodiment, the nucleic acid construct is a variant of SEQ ID NO: 33. In another embodiment, the nucleic acid construct is an isomorph of SEQ ID NO: 33. In another embodiment, the nucleic acid construct is a homolog of SEQ ID NO: 33.

In one embodiment, disclosed herein is a bivalent, trivalent, or quadravalent recombinant Listeria strain expressing two, three or fourheterologous antigens fused to an N-terminal or truncated or detoxified Listeriolysin O protein (LLO), a truncated ActA protein, or a PEST amino acid sequence disclosed herein.

In one embodiment, disclosed herein is a trivalent, or quadravalent recombinant Listeria strain expressing three heterologous antigens fused to an N-terminal or truncated or detoxified Listeriolysin O protein, a truncated ActA protein, or a PEST amino acid sequence disclosed herein.

In one embodiment, disclosed herein is a quadravalent recombinant Listeria strain expressing four heterologous antigens fused to an N-terminal or truncated or detoxified Listeriolysin O protein (LLO). In another embodiment, the PEST-containing polypeptide is an N-terminal, a truncated ActA protein, or a PEST amino acid sequence disclosed herein. In another embodiment, the heterologous antigens disclosed herein are individually fused to an N-terminal or truncated LLO or truncated ActA or PEST sequence on the N-terminus or the C-terminus of said LLO, truncated ActA or PEST sequence. In another embodiment, each of the heterologous antigens disclosed herein are individually fused to an N-terminal or truncated LLO, truncated ActA or PEST sequence on the N-terminus or the C-terminus of said LLO, truncated ActA, or PEST sequence.

In one embodiment, a bivalent, trivalent, or quadravalent recombinant Listeria strains disclosed herein express at least one heterologous antigen from an open reading frame in an extrachromosomal plasmid or episome. In another embodiment, the bivalent, trivalent, or quadravalent recombinant Listeria strains disclosed herein express at least one heterologous antigen from an open reading frame for at least one extrachromosomal plasmid or episome. In another embodiment, the bivalent recombinant Listeria strains disclosed herein express two heterologous antigens each from an open reading frame of two extrachromosomal plasmids or episomes. In another embodiment, the trivalent recombinant Listeria strains disclosed herein express three heterologous antigens each from an open reading frame of three extrachromosomal plasmids or episomes. In another embodiment, the quadravalent recombinant Listeria strains disclosed herein express four heterologous antigens each from an open reading frame of four extrachromosomal plasmids or episomes.

In another embodiment, a bivalent, trivalent, or quadravalent recombinant Listeria strains disclosed herein express at least one heterologous antigen from an open reading frame in the genome of the Listeria. In another embodiment, the bivalent, trivalent, or quadravalent recombinant Listeria strains disclosed herein express at least one heterologous antigen from both, an extrachromosomal plasmid or episome, and from the genome of a Listeria disclosed herein. In another embodiment, each heterologous antigen is expressed in a fusion protein with an N-terminal or truncated or detoxified Listeriolysin O protein (LLO). In another embodiment, the PEST-containing polypeptide is, an N-terminal or a truncated ActA protein. In another embodiment, the PEST-containing peptide is a PEST amino acid sequence disclosed herein.

In one embodiment, bivalent and multivalent recombinant Listeria encompassed by the present invention include those described in US Pub. No. 2011/0129499, and in US Pub No. 2012/0135033, both of which are incorporated by reference in their entirety herein.

In another embodiment, the subject is at risk for developing an HPV-mediated carcinogenesis (e.g. a cervical cancer). In another embodiment, the subject is HPV-positive.

In another embodiment, the subject exhibits cervical intraepithelial neoplasia. In another embodiment, the subject exhibits a squamous intraepithelial lesion. In another embodiment, the subject exhibits a dysplasia in the cervix.

In one embodiment, the heterologous antigen is any tumor associated antigen known in the art and disclosed herein. In another embodiment, the heterologous antigen is an autoimmune antigen. In another embodiment, the heterologous antigen is an infectious disease antigen. In another embodiment, the heterologous antigen is an HPV-related antigen.

The HPV that is the target of methods of the present invention is, in another embodiment, an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the present invention provides a method of vaccinating a human subject against a heterologus antigen, the method comprising the step of administering intravenously to the human subject a recombinant Listeria strain comprising or expressing the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of vaccinating a human subject against a heterologus antigen, the method comprising the step of administering intravenously to the human subject an immunogenic composition, comprising a fusion of a first peptide to the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of vaccinating a human subject against a heterologus antigen, the method comprising the step of administering intravenously to the human subject a recombinant Listeria strain comprising a recombinant polypeptide, the recombinant polypeptide comprising a first peptide fused to the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of inducing a CTL response in a human subject against a heterologus antigen, the method comprising the step of administering to the human subject a recombinant Listeria strain comprising or expressing the heterologus antigen, thereby inducing a CTL response in a human subject against a heterologus antigen. In another embodiment, the step of administering is intravenous administration.

As disclosed herein, recombinant Listeria strains expressing LLO-antigen fusions induce anti-tumor immunity (Example 1), elicit antigen-specific T cell proliferation (Example 2), generate antigen-specific, and tumor-infiltrating T cells (Example 3). Thus, vaccines of the present invention are efficacious at inducing immune responses against E7 and E6.

In another embodiment, the present invention provides a method for inducing a regression of a cancer in a subject, comprising the step of administering to the subject the recombinant Listeria strain disclosed herein

In another embodiment, the present invention provides a method for reducing an incidence of relapse of a cancer in a subject, comprising the step of administering to the subject the recombinant Listeria strain disclosed herein.

In another embodiment, the present invention provides a method for suppressing a formation of a tumor in a subject, comprising the step of administering to the subject the recombinant Listeria strain disclosed herein.

In another embodiment, the present invention provides a method for inducing a remission of a cancer in a subject, comprising the step of administering to the subject the recombinant Listeria strain disclosed herein.

In another embodiment, the present invention provides a method for impeding a growth of a tumor in a human subject, comprising the step of administering to the subject the recombinant Listeria strain disclosed herein.

In another embodiment, the present invention provides a method for reducing a size of a tumor in a subject, comprising the step of administering to the subject the recombinant Listeria strain disclosed herein.

In one embodiment, the disease is an infectious disease, an autoimmune disease, a respiratory disease, a pre-cancerous condition or a cancer.

It will be well appreciated by the skilled artisan that the term “pre-cancerous condition” may encompass dysplasias, preneoplastic nodules; macroregenerative nodules (MRN); low-grade dysplastic nodules (LG-DN); high-grade dysplastic nodules (HG-DN); biliary epithelial dysplasia; foci of altered hepatocytes (FAH); nodules of altered hepatocytes (NAH); chromosomal imbalances; aberrant activation of telomerase; re-expression of the catalytic subunit of telomerase; expression of endothelial cell markers such as CD31, CD34, and BNH9 (see, e.g., Terracciano and Tomillo (2003) Pathologica 95:71-82; Su and Bannasch (2003) Toxicol. Pathol. 31:126-133; Rocken and Carl-McGrath (2001) Dig. Dis. 19:269-278; Kotoula, et al. (2002) Liver 22:57-69; Frachon, et al. (2001) J. Hepatol. 34:850-857; Shimonishi, et al. (2000) J. Hepatobiliary Pancreat. Surg. 7:542-550; Nakanuma, et al. (2003) J. Hepatobiliary Pancreat. Surg. 10:265-281). Methods for diagnosing cancer and dysplasia are disclosed (see, e.g., Riegler (1996) Semin. Gastrointest. Dis. 7:74-87; Benvegnu, et al. (1992) Liver 12:80-83; Giannini, et al. (1987) Hepatogastroenterol. 34:95-97; Anthony (1976) Cancer Res. 36:2579-2583).

In one embodiment, an infectious disease is one caused by, but not limited to, any one of the following pathogens: BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola , Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E.coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas.

In another embodiment, the disease disclosed herein is a respiratory or inflammatory disease. In another embodiment, the respiratory or inflammatory disease is chronic obstructive pulmonary disease (COPD). In another embodiment, the disease is asthma.

In one embodiment, live attenuated Listeria strains are capable of alleviating asthma symptoms without co-administration of other therapeutic agents, such as anti-inflammatory agents or bronchodilators. In another embodiment, the methods disclosed herein further comprise the step of co-administering to a subject the live attenuated Listeria strain and one or more therapeutic agents. In another embodiment, the therapeutic agent is an anti-asthmatic agent. In another embodiment, the agent is an anti-inflammatory agent, a non-steroidal anti-inflammatory agent, an antibiotic, an antichlolinerginc agent, a bronchodilator, a corticosteroid, a short-acting beta-agonist, a long-acting beta-agonist, combination inhalers, an antihistamine, or combinations thereof.

In one embodiment, a disease disclosed herein is a cancer or a tumor. In one embodiment, the tumor is cancerous. In another embodiment, the cancer is breast cancer. In another embodiment, the cancer is a cervical cancer. In another embodiment, the cancer is a Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is lung cancer. In another embodiment, the cancer is vaginal cancer. In another embodiment, the cancer is colorectal cancer. In another embodiment, the cancer is esophageal cancer. The cervical tumor targeted by methods of the present invention is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In another embodiment, the compositions disclosed herein are useful for inducing an immune response against, preventing or treating a anal intracelluar neoplasia in a subject. In another embodiment, the compositions disclosed herein are useful for inducing an immune response against, preventing or treating a vaginal intracelluar neoplasia in a subject.

The cervical tumor targeted by methods of the present invention is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In one embodiment, an antigen may be foreign, that is, heterologous to the host and is referred to as a “heretologous antigen” herein. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen. In addition, a heterologous antigen may encompass an infectious disease antigen.

In one embodiment, the antigen disclosed herein is a heterologous tumor antigen, which is also referred to herein as “tumor antigen” “antigenic polypeptide,” or “foreign antigen.” In another embodiment, the antigen disclosed herein is a self-antigen.

It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein” , “heterologous protein antigen” “protein antigen”, “antigen” and the like.

In one embodiment, the antigen is Human Papilloma Virus-E7 (HPV-E7) antigen, which in one embodiment, is from HPV16 (in one embodiment, GenBank Accession No. AAD33253) and in another embodiment, from HPV18 (in one embodiment, GenBank Accession No. P06788). In another embodiment, the antigenic polypeptide is HPV-E6, which in one embodiment, is from HPV16 (in one embodiment, GenBank Accession No. AAD33252, AAM51854, AAM51853, or AAB67615) and in another embodiment, from HPV18 (in one embodiment, GenBank Accession No. P06463). In another embodiment, the antigenic polypeptide is a Her/2-neu antigen. In another embodiment, the antigenic polypeptide is Prostate Specific Antigen (PSA) (in one embodiment, GenBank Accession No. CAD30844, CAD54617, AAA58802, or NP_001639). In another embodiment, the antigenic polypeptide is Stratum Corneum Chymotryptic Enzyme (SCCE) antigen (in one embodiment, GenBank Accession No. AAK69652, AAK69624, AAG33360, AAF01139, or AAC37551). In another embodiment, the antigenic polypeptide is Wilms tumor antigen 1, which in another embodiment is WT-1 Telomerase (GenBank Accession. No. P49952, P22561, NP_659032, CAC39220.2, or EAW68222.1). In another embodiment, the antigenic polypeptide is hTERT or Telomerase (GenBank Accession. No. NM003219 (variant 1), NM198255 (variant 2), NM 198253 (variant 3), or NM 198254 (variant 4). In another embodiment, the antigenic polypeptide is Proteinase 3 (in one embodiment, GenBank Accession No. M29142, M75154, M96839, X55668, NM 00277, M96628 or X56606). In another embodiment, the antigenic polypeptide is Tyrosinase Related Protein 2 (TRP2) (in one embodiment, GenBank Accession No. NP_001913, ABI73976, AAP33051, or Q95119). In another embodiment, the antigenic polypeptide is High Molecular Weight Melanoma Associated Antigen (HMW-MAA) (in one embodiment, GenBank Accession No. NP_001888, AAI28111, or AAQ62842). In another embodiment, the antigenic polypeptide is Testisin (in one embodiment, GenBank Accession No. AAF79020, AAF79019, AAG02255, AAK29360, AAD41588, or NP_659206). In another embodiment, the antigenic polypeptide is NY-ESO-1 antigen (in one embodiment, GenBank Accession No. CAA05908, P78358, AAB49693, or NP_640343). In another embodiment, the antigenic polypeptide is PSCA (in one embodiment, GenBank Accession No. AAH65183, NP_005663, NP_082492, 043653, or CAB97347). In another embodiment, the antigenic polypeptide is Interleukin (IL) 13 Receptor alpha (in one embodiment, GenBank Accession No. NP_000631, NP_001551, NP_032382, NP_598751, NP_001003075, or NP_999506). In another embodiment, the antigenic polypeptide is Carbonic anhydrase IX (CAIX) (in one embodiment, GenBank Accession No. CAI13455, CAI10985, EAW58359, NP_001207, NP_647466, or NP_001101426). In another embodiment, the antigenic polypeptide is carcinoembryonic antigen (CEA) (in one embodiment, GenBank Accession No. AAA66186, CAA79884, CAA66955, AAA51966, AAD15250, or AAA51970.). In another embodiment, the antigenic polypeptide is MAGE-A (in one embodiment, GenBank Accession No. NP_786885, NP_786884, NP_005352, NP_004979, NP_005358, or NP_005353). In another embodiment, the antigenic polypeptide is survivin (in one embodiment, GenBank Accession No. AAC51660, AAY15202, ABF60110, NP_001003019, or NP_001082350). In another embodiment, the antigenic polypeptide is GP100 (in one embodiment, GenBank Accession No. AAC60634, YP_655861, or AAB31176). In another embodiment, the antigenic polypeptide is any other antigenic polypeptide known in the art. In another embodiment, the antigenic peptide of the compositions and methods of the present invention comprise an immunogenic portion of the antigenic polypeptide.

In another embodiment, the antigen is HPV-E6. In another embodiment, the antigen is telomerase (TERT). In another embodiment, the antigen is LMP-1. In another embodiment, the antigen is p53. In another embodiment, the antigen is mesothelin. In another embodiment, the antigen is EGFRVIII. In another embodiment, the antigen is carboxic anhydrase IX (CAIX). In another embodiment, the antigen is PSMA. In another embodiment, the antigen is HMW-MAA. In another embodiment, the antigen is HIV-1 Gag. In another embodiment, the antigen is Tyrosinase related protein 2. In another embodiment, the antigen is selected from HPV-E7, HPV-E6, Her-2, HIV-1 Gag, LMP-1, p53, PSMA, carcinoembryonic antigen (CEA), LMP-1,kallikrein-related peptidase 3 (KLK3), KLK9, Muc, Tyrosinase related protein 2, Muc1, FAP, IL-13R alpha 2, PSA (prostate-specific antigen), gp-100, heat-shock protein 70 (HSP-70), beta-HCG, EGFR-III, Granulocyte colony-stimulating factor (G-CSF), Angiogenin, Angiopoietin-1, Del-1, Fibroblast growth factors: acidic (aFGF) or basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF), VEGFR, VEGFR2 (KDR/FLK-1) or a fragment thereof, FLK-1 or an epitope thereof, FLK-E1, FLK-E2, FLK-I1, endoglin or a fragment thereof, Neuropilin 1 (NRP-1), Angiopoietin 1 (Ang1), Tie2, Platelet-derived growth factor (PDGF), Platelet-derived growth factor receptor (PDGFR), Transforming growth factor-beta (TGF-β), endoglin, TGF-β receptors, monocyte chemotactic protein-1 (MCP-1), VE-cadherin, CD31, ephrin, ICAM-1, V-CAM-1, VAP-1, E-selectin, plasminogen activators, plasminogen activator inhibitor-1, Nitric oxide synthase (NOS), COX-2, AC133, or Id1/Id3, Angiopoietin 3, Angiopoietin 4, Angiopoietin 6, CD105, EDG, HHT1, ORW, ORW1 or a TGFbeta co-receptor, or a combination thereof. In another embodiment, the antigen is a chimeric Her2/neu antigen as disclosed in US Patent Application Publication No. 2011/0142791, which is incorporated by reference herein in its entirety. The use of fragments of antigens disclosed herein is also encompassed by the present invention.

In another embodiment, the heterologous tumor antigen disclosed herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, a MART1 antigen associated with melanoma, or the PSA antigen associated with prostate cancer. In another embodiment, the antigen for the compositions and methods disclosed herein are melanoma-associated antigens, which in one embodiment are TRP-2, MAGE-1, MAGE-3, gp-100, tyrosinase, HSP-70, beta-HCG, or a combination thereof. It is to be understood that a skilled artisan would be able to use any heterologous antigen not mentioned herein but known in the art for use in the methods and compositions disclosed herein. It is also to be understood that the present invention provides, but is not limited by, an attenuated Listeria comprising a nucleic acid that encodes at least one of the antigens disclosed herein. The present invention encompasses nucleic acids encoding mutants, muteins, splice variants, fragments, truncated variants, soluble variants, extracellular domains, intracellular domains, mature sequences, and the like, of the disclosed antigens. Disclosed are nucleic acids encoding epitopes, oligo- and polypeptides of these antigens. Also disclosed are codon optimized embodiments, that is, optimized for expression in Listeria. The cited references, GenBank Acc. Nos., and the nucleic acids, peptides, and polypeptides disclosed herein, are all incorporated herein by reference in their entirety. In another embodiment, the selected nucleic acid sequence can encode a full length or a truncated gene, a fusion or tagged gene, and can be a cDNA, a genomic DNA, or a DNA fragment, preferably, a cDNA. It can be mutated or otherwise modified as desired. These modifications include codon optimizations to optimize codon usage in the selected host cell or bacteria, i.e. Listeria. The selected sequence can also encode a secreted, cytoplasmic, nuclear, membrane bound or cell surface polypeptide.

In one embodiment, vascular endothelial growth factor (VEGF) is an important signaling protein involved in both vasculogenesis (the formation of the embryonic circulatory system) and angiogenesis (the growth of blood vessels from pre-existing vasculature). In one embodiment, VEGF activity is restricted mainly to cells of the vascular endothelium, although it does have effects on a limited number of other cell types (e.g. stimulation monocyte/macrophage migration). In vitro, VEGF has been shown to stimulate endothelial cell mitogenesis and cell migration. VEGF also enhances microvascular permeability and is sometimes referred to as vascular permeability factor.

In one embodiment, all of the members of the VEGF family stimulate cellular responses by binding to tyrosine kinase receptors (the VEGFRs) on the cell surface, causing them to dimerize and become activated through transphosphorylation. The VEGF receptors have an extracellular portion consisting of 7 immunoglobulin-like domains, a single transmembrane spanning region and an intracellular portion containing a split tyrosine-kinase domain.

In one embodiment, VEGF-A is a VEGFR-2 (KDR/Flk-1) ligand as well as a VEGFR-1 (Flt-1) ligand. In one embodiment, VEGFR-mediates almost all of the known cellular responses to VEGF. The function of VEGFR-1 is less well defined, although it is thought to modulate VEGFR-2 signaling, in one embodiment, via sequestration of VEGF from VEGFR-2 binding, which in one embodiment, is particularly important during vasculogenesis in the embryo. In one embodiment, VEGF-C and VEGF-D are ligands of the VEGFR-3 receptor, which in one embodiment, mediates lymphangiogenesis.

In one embodiment, the compositions of the present invention comprise a VEGF receptor or a fragment thereof, which in one embodiment, is a VEGFR-2 and, in another embodiment, a VEGFR-1, and, in another embodiment, VEGFR-3.

In one embodiment, vascular Endothelial Growth Factor Receptor 2 (VEGFR2) is highly expressed on activated endothelial cells (ECs) and participates in the formation of new blood vessels. In one embodiment, VEGFR2 binds all 5 isoforms of VEGF. In one embodiment, signaling of VEGF through VEGFR2 on ECs induces proliferation, migration, and eventual differentiation. In one embodiment, the mouse homologue of VEGFR2 is the fetal liver kinase gene-1 (Flk-1), which is a strong therapeutic target, and has important roles in tumor growth, invasion, and metastasis. In one embodiment, VEGFR2 is also referred to as kinase insert domain receptor (a type III receptor tyrosine kinase) (KDR), cluster of differentiation 309 (CD309), FLK1, Ly73, Krd-1, VEGFR, VEGFR-2, or 6130401C07.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens El and E2 from type HPV-16, -18, -31, -33, -35 or -45 human papilloma viruses, the tumor antigens CEA, the ras protein, mutated or otherwise, the p53 protein, mutated or otherwise, Mucl, or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangiti s, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment, an HPV E6 antigen is utilized instead of or in addition to an E7 antigen in a method of the present invention for treating, protecting against, or inducing an immune response against a cervical cancer.

In another embodiment, an ActA protein fragment is utilized instead of or in addition to an LLO fragment in a method of the present invention for treating, protecting against, or inducing an immune response against a cervical cancer.

In another embodiment, a PEST amino acid sequence-containing protein fragment is utilized instead of or in addition to an LLO fragment in a method of the present invention for treating, protecting against, or inducing an immune response against a cervical cancer.

In another embodiment, the present invention provides an immunogenic composition comprising a recombinant Listeria of the present invention. In another embodiment, the immunogenic composition of methods and compositions of the present invention comprises a recombinant vaccine vector of the present invention. In another embodiment, the immunogenic composition comprises a plasmid of the present invention. In another embodiment, the immunogenic composition comprises an adjuvant. In one embodiment, a vector of the present invention may be administered as part of a vaccine composition.

In another embodiment, a vaccine of the present invention is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the present invention provides a method for inducing an anti-E7 cytotoxic T cell (CTL) response in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing an anti-E7 CTL response in a human subject. In another embodiment, the recombinant Listeria strain comprises a plasmid that encodes the recombinant polypeptide. In another embodiment, the method further comprises the step of boosting the subject with a recombinant Listeria strain of the present invention. In another embodiment, the method further comprises the step of boosting the subject with an immunogenic composition comprising an E7 antigen. In another embodiment, the method further comprises the step of boosting the subject with an immunogenic composition that directs a cell of the subject to express an E7 antigen. In another embodiment, the CTL response is capable of therapeutic efficacy against an HPV-mediated disease, disorder, or symptom. In another embodiment, the CTL response is capable of prophylactic efficacy against an HPV-mediated disease, disorder, or symptom.

In another embodiment, the present invention provides a method of treating or ameliorating an HPV-mediated disease, disorder, or symptom in a subject, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby treating or ameliorating an HPV-mediated disease, disorder, or symptom in a subject. In another embodiment, the subject is a human subject. In another embodiment, the subject is a non-human mammal. In another embodiment, the subject is any other type of subject known in the art.

The HPV causing the disease, disorder, or symptom is, in another embodiment, an HPV 16. In another embodiment, the HPV is an HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the HPV-mediated disease, disorder, or symptom is genital warts. In another embodiment, the HPV-mediated disease, disorder, or symptom is non-genital warts. In another embodiment, the HPV-mediated disease, disorder, or symptom is a respiratory papilloma. In another embodiment, the HPV-mediated disease, disorder, or symptom is any other HPV-mediated disease, disorder, or symptom known in the art.

In another embodiment, an HPV E6 antigen is utilized instead of or in addition to an E7 antigen in a method of the present invention for treating or ameliorating an HPV-mediated disease, disorder, or symptom.

In another embodiment, an ActA protein fragment is utilized instead of or in addition to an LLO fragment in a method of the present invention for treating or ameliorating an HPV-mediated disease, disorder, or symptom.

In another embodiment, a PEST amino acid sequence-containing protein fragment is utilized instead of or in addition to an LLO fragment in a method of the present invention for treating or ameliorating an HPV-mediated disease, disorder, or symptom.

In another embodiment, an HPV E6 antigen is utilized instead of or in addition to an E7 antigen in a method of the present invention for treating or ameliorating an HPV-mediated disease, disorder, or symptom.

The antigen of methods and compositions of the present invention is, in another embodiment, an HPV E7 protein. In another embodiment, the antigen is an HPV E6 protein. In another embodiment, the antigen is any other HPV protein known in the art.

“E7 antigen” refers, in another embodiment, to an E7 protein. In another embodiment, the term refers to an E7 fragment. In another embodiment, the term refers to an E7 peptide. In another embodiment, the term refers to any other type of E7 antigen known in the art.

The E7 protein of methods and compositions of the present invention is, in another embodiment, an HPV 16 E7 protein. In another embodiment, the E7 protein is an HPV-18 E7 protein. In another embodiment, the E7 protein is an HPV-31 E7 protein. In another embodiment, the E7 protein is an HPV-35 E7 protein. In another embodiment, the E7 protein is an HPV-39 E7 protein. In another embodiment, the E7 protein is an HPV-45 E7 protein. In another embodiment, the E7 protein is an HPV-51 E7 protein. In another embodiment, the E7 protein is an HPV-52 E7 protein. In another embodiment, the E7 protein is an HPV-58 E7 protein. In another embodiment, the E7 protein is an E7 protein of a high-risk HPV type. In another embodiment, the E7 protein is an E7 protein of a mucosal HPV type.

“E6 antigen” refers, in another embodiment, to an E6 protein. In another embodiment, the term refers to an E6 fragment. In another embodiment, the term refers to an E6 peptide. In another embodiment, the term refers to any other type of E6 antigen known in the art.

The E6 protein of methods and compositions of the present invention is, in another embodiment, an HPV 16 E6 protein. In another embodiment, the E6 protein is an HPV-18 E6 protein. In another embodiment, the E6 protein is an HPV-31 E6 protein. In another embodiment, the E6 protein is an HPV-35 E6 protein. In another embodiment, the E6 protein is an HPV-39 E6 protein. In another embodiment, the E6 protein is an HPV-45 E6 protein. In another embodiment, the E6 protein is an HPV-51 E6 protein. In another embodiment, the E6 protein is an HPV-52 E6 protein. In another embodiment, the E6 protein is an HPV-58 E6 protein. In another embodiment, the E6 protein is an E6 protein of a high-risk HPV type. In another embodiment, the E6 protein is an E6 protein of a mucosal HPV type.

The immune response induced by methods and compositions of the present invention is, in another embodiment, a T cell response. In another embodiment, the immune response comprises a T cell response. In another embodiment, the response is a CD8⁺ T cell response. In another embodiment, the response comprises a CD8⁺ T cell response.

In one embodiment, compositions of the present invention induce a strong innate stimulation of interferon-gamma, which in one embodiment, has anti-angiogenic properties. In one embodiment, a Listeria of the present invention induces a strong innate stimulation of interferon-gamma, which in one embodiment, has anti-angiogenic properties (Dominiecki et al., Cancer Immunol Immunother. 2005 May; 54(5):477-88. Epub 2004 Oct 6, incorporated herein by reference in its entirety; Beatty and Paterson, J Immunol. 2001 Feb. 15; 166(4):2276-82, incorporated herein by reference in its entirety). In another embodiment, methods of the present invention increase a level of interferon-gamma producing cells. In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, compositions of the present invention induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

In another embodiment the immune response induced by methods and compositions of the present invention is suppression of programmed cell death receptor-1 ligand 1 (PD-L1) expression in the target tumor cells. In another embodiment, the immune response comprises increased level of programmed cell death receptor-1 (PD-1) expressing immune cells within tumor. In another embodiment, the immune response comprises increase in ratio of the level of PD-1 expression to PD-L1 expression. In another embodiment, the immune response comprises inhibition of tumor PD-L1-mediated immunosuppression.

In another embodiment, the administration of compositions of the present invention induces robust systemic antigen-specific immunity. In another embodiment, the administration of compositions of the present invention induces epitope spreading. In another embodiment, the administration of compositions of the present invention induces broad-based response to self-derived tumor antigens. In another embodiment the immune response induced by methods and compositions of the present invention comprises improvement of the overall balance of suppressor and effector immune cells in the tumor microenvironment (TME). In another embodiment the immune response induced by methods and compositions of the present invention comprises improvement in the systemic balance of suppressor and effector immunocytes.

In one embodiment, compositions and methods of use thereof as disclosed herein generate effector T cells that are able to infiltrate the tumor, destroy tumor cells and eradicate the disease. In another embodiment, methods of use of this invention increase umore infilatration by T effector cells. In another embodiment, T effector cells comprise CD8+ T cells. In another embodiment, T effector cells comprise CD4+ T cells.

In one embodiment, tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as disclosed herein increase TILs, up-regulate adhesion molecules (in one embodiment, ICAM-1, V-CAM-1, VAP-1, E-selectin, or a combination thereof), up-regulate pro-inflammatory cytokines (in one embodiment, IFN-γ, TNF-α, IL-1, or a combination thereof), or a combination thereof.

The N-terminal LLO protein fragment of methods and compositions of the present invention comprises, in another embodiment, SEQ ID No: 2. In another embodiment, the fragment comprises an LLO signal peptide. In another embodiment, the fragment comprises SEQ ID No: 2. In another embodiment, the fragment consists approximately of SEQ ID No: 2. In another embodiment, the fragment consists essentially of SEQ ID No: 2. In another embodiment, the fragment corresponds to SEQ ID No: 2. In another embodiment, the fragment is homologous to SEQ ID No: 2. In another embodiment, the fragment is homologous to a fragment of SEQ ID No: 2. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the present invention. In another embodiment, fusion of an E7 or E6 antigen to any ΔLLO, including the PEST amino acid AA sequence, SEQ ID NO: 1, enhances cell mediated and anti-tumor immunity of the antigen.

The LLO protein utilized to construct vaccines of the present invention has, in another embodiment, the sequence:

MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYPNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDPEGNEIVQH
KNWSENNKSKLAHFTSSIYLPGNARNINVYAKECTGLAWEWWRTVIDDRN
LPLVKNRNISIWGTTLYPKYSNKVDNPIE

(GenBank Accession No. P13128; SEQ ID NO: 3; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention.

In another embodiment, the N-terminal fragment of an LLO protein utilized in compositions and methods of the present invention has the sequence:

(SEQ ID NO: 2)

MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 4)

MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In another embodiment, “truncated LLO” or “ΔLLO” refers to a fragment of LLO that comprises the PEST amino acid domain. In another embodiment, the terms refer to an LLO fragment that comprises a PEST sequence.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly.

In another embodiment, the LLO fragment is any other LLO fragment known in the art.

In another embodiment, the recombinant Listeria strain is administered to the human subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU. In another embodiment, the recombinant Listeria strain is administered to the human subject at a dose of from about 1×10⁶ to about 3.31×10¹⁰ CFU. In another embodiment, the dose is from about 1×10⁶ to about 1×10¹⁰ CFU. In another embodiment, the dose is from about 1×10⁶ to about 1×10⁹ CFU. In another embodiment, the dose is from about 1×10⁶ to about 1×10⁷ CFU. In another embodiment, the dose is from about 1×10⁶ to about 1×10⁸ CFU. In another embodiment, the dose is from about 1×10⁷ CFU to about 1×10⁹ CFU. In another embodiment, the dose is from about 1×10⁸ CFU to about 1×10⁹ CFU.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In another embodiment, the recombinant polypeptide of methods of the present invention is expressed by the recombinant Listeria strain. In another embodiment, the expression is mediated by a nucleotide molecule carried by the recombinant Listeria strain.

In another embodiment, the recombinant Listeria strain expresses the recombinant polypeptide by means of a plasmid that encodes the recombinant polypeptide. In another embodiment, the plasmid comprises a gene encoding a bacterial transcription factor.

In another embodiment, the plasmid encodes a Listeria transcription factor. In another embodiment, the transcription factor is PrfA. In another embodiment, the PrfA is a mutant PrfA. In another embodiment, the PrfA contains a D133V amino acid mutation. In another embodiment, the transcription factor is any other transcription factor known in the art.

In another embodiment, the plasmid comprises a gene encoding a metabolic enzyme. In another embodiment, the metabolic enzyme is a bacterial metabolic enzyme. In another embodiment, the metabolic enzyme is a Listerial metabolic enzyme. In another embodiment, the metabolic enzyme is an amino acid metabolism enzyme. In another embodiment, the amino acid metabolism gene is involved in a cell wall synthesis pathway. In another embodiment, the metabolic enzyme is the product of a D-amino acid aminotransferase gene (dat). In another embodiment, the metabolic enzyme is the product of an alanine racemase gene (dal). In another embodiment, the metabolic enzyme is any other metabolic enzyme known in the art.

In another embodiment, a method of present invention further comprises the step of boosting the human subject with a recombinant Listeria strain of the present invention. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster.

In another embodiment, a method of present invention further comprises the step of inoculating the human subject with an immunogenic composition comprising the E7 antigen. In another embodiment, the immunogenic composition comprises a recombinant E7 protein or fragment thereof. In another embodiment, the immunogenic composition comprises a nucleotide molecule expressing a recombinant E7 protein or fragment thereof. In another embodiment, the non-Listerial inoculation is administered after the Listerial inoculation. In another embodiment, the non-Listerial inoculation is administered before the Listerial inoculation.

“Boosting” refers, in another embodiment, to administration of an additional vaccine dose to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

The present invention provides a number of Listerial species and strains for making or engineering an attenuated Listeria of the present invention. In one embodiment, the Listeria strain is L. monocytogenes 10403S wild type (see Bishop and Hinrichs (1987) J. Immunol. 139: 2005-2009; Lauer, et al. (2002) J. Bact. 184: 4177-4186.) In another embodiment, the Listeria strain is L. monocytogenes DP-L4056 (phage cured) (see Lauer, et al. (2002) J. Bact. 184: 4177-4186). In another embodiment, the Listeria strain is L. monocytogenes DP-L4027, which is phage cured and deleted in the hly gene (see Lauer, et al. (2002) J. Bact. 184: 4177-4186; Jones and Portnoy (1994) Infect. Immunity 65: 5608-5613.). In another embodiment, the Listeria strain is L. monocytogenes DP-L4029, which is phage cured, deleted in ActA (see Lauer, et al. (2002) J. Bact. 184: 4177-4186; Skoble, et al. (2000) J. Cell Biol. 150: 527-538). In another embodiment, the Listeria strain is L. monocytogenes DP-L4042 (delta PEST) (see Brockstedt, et al. (2004) Proc. Natl. Acad. Sci. USA 101: 13832-13837; supporting information). In another embodiment, the Listeria strain is L. monocytogenes DP-L4097 (LLO-S44A) (see Brockstedt, et al. (2004) Proc. Natl. Acad. Sci. USA 101: 13832-13837; supporting information). In another embodiment, the Listeria strain is L. monocytogenes DP-L4364 (delta lplA; lipoate protein ligase) (see Brockstedt, et al. (2004) Proc. Natl. Acad. Sci. USA 101: 13832-13837; supporting information). In another embodiment, the Listeria strain is L. monocytogenes DP-L4405 (delta inlA) (see Brockstedt, et al. (2004) Proc. Natl. Acad. Sci. USA 101: 13832-13837; supporting information). In another embodiment, the Listeria strain is L. monocytogenes DP-L4406 (delta inlB) (see Brockstedt, et al. (2004) Proc. Natl. Acad. Sci. USA 101: 13832-13837; supporting information). In another embodiment, the Listeria strain is L. monocytogenes CS-L0001 (delta ActA-delta inlB) (see Brockstedt, et al. (2004) Proc. Natl. Acad. Sci. USA 101: 13832-13837; supporting information). In another embodiment, the Listeria strain is L. monocytogenes CS-L0002 (delta ActA-delta lplA) (see Brockstedt, et al. (2004) Proc. Natl. Acad. Sci. USA 101: 13832-13837; supporting information). In another embodiment, the Listeria strain is L. monocytogenes CS-L0003 (L461T-delta lplA) (see Brockstedt, et al. (2004) Proc. Natl. Acad. Sci. USA 101: 13832-13837; supporting information). In another embodiment, the Listeria strain is L. monocytogenes DP-L4038 (delta ActA-LLO L461T) (see Brockstedt, et al. (2004) Proc. Natl. Acad. Sci. USA 101: 13832-13837; supporting information). In another embodiment, the Listeria strain is L. monocytogenes DP-L4384 (S44A-LLO L461T) (see Brockstedt, et al. (2004) Proc. Natl. Acad. Sci. USA 101: 13832-13837; supporting information). In another embodiment, the Listeria strain is L. monocytogenes. Mutation in lipoate protein (see O'Riordan, et al. (2003) Science 302: 462-464). In another embodiment, the Listeria strain is L. monocytogenes DP-L4017 (10403S hly (L461T), having a point mutation in hemolysin gene (see U.S. Provisional Pat. Appl. Ser. No. 60/490,089 filed Jul. 24, 2003). In another embodiment, the Listeria strain is L. monocytogenes EGD (see GenBank Acc. No. AL591824). In another embodiment, the Listeria strain is L. monocytogenes EGD-e (see GenBank Acc. No. NC_003210. ATCC Acc. No. BAA-679). In another embodiment, the Listeria strain is L. monocytogenes DP-L4029 deleted in uvrAB (see U.S. Provisional Pat. Appl. Ser. No. 60/541,515 filed Feb. 2, 2004; U.S. Provisional Pat. Appl. Ser. No. 60/490,080 filed Jul. 24, 2003). In another embodiment, the Listeria strain is L. monocytogenes ActA-/in1B-double mutant (see ATCC Acc. No. PTA-5562). In another embodiment, the Listeria strain is L. monocytogenes lplA mutant or hly mutant (see U.S. Pat. Applic. No. 20040013690 of Portnoy, et. al). In another embodiment, the Listeria strain is L. monocytogenes DAL/DAT double mutant. (see U.S. Pat. Applic. No. 20050048081 of Frankel and Portnoy. The present invention encompasses reagents and methods that comprise the above Listeria strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, uptake by a host cell. The present invention is not to be limited by the particular strains disclosed above.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein (e.g. in Example 12). In another embodiment, the passaging is performed by any other method known in the art.

In another embodiment, the recombinant Listeria strain utilized in methods of the present invention has been stored in a frozen cell bank. In another embodiment, the recombinant Listeria strain has been stored in a lyophilized cell bank.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods of the present invention is from a batch of vaccine doses.

In another embodiment, a recombinant Listeria strain utilized in methods of the present invention is from a frozen or lyophilized stock produced by methods disclosed in U.S. Pat. No. 8,114,414, which is incorporated by reference herein.

In another embodiment, a peptide of the present invention is a fusion peptide. In another embodiment, “fusion peptide” refers to a peptide or polypeptide comprising 2 or more proteins linked together by peptide bonds or other chemical bonds. In another embodiment, the proteins are linked together directly by a peptide or other chemical bond. In another embodiment, the proteins are linked together with 1 or more AA (e.g. a “spacer”) between the 2 or more proteins.

In another embodiment, a vaccine of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In another embodiment, a nucleotide of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto.

An N-terminal fragment of an ActA protein utilized in methods and compositions of the present invention has, in another embodiment, the sequence set forth in SEQ ID NO: 5:

MRAMMVVFITANCITINPDIIFAATDSEDSSLNTDEWEEEKTEEQPSEVN
TGPRYETAREVSSRDIKELEKSNKVRNTNKADLIAMLKEKAEKGPNINNN
NSEQTENAAINEEASGADRPAIQVERRHPGLPSDSAAEIKKRRKAIASSD
SELESLTYPDKPTKVNKKKVAKESVADASESDLDSSMQSADESSPQPLKA
NQQPFFPKVFKKIKDAGKWVRDKIDENPEVKKAIVDKSAGLIDQLLTKKK
SEEVNASDFPPPPTDEELRLALPETPMLLGFNAPATSEPSSFEFPPPPTD
EELRLALPETPMLLGFNAPATSEPSSFEFPPPPTEDELEIIRETASSLDS
SFTRGDLASLRNAINRHSQNFSDFPPIPTEEELNGRGGRP.

In another embodiment, the ActA fragment comprises the sequence set forth in SEQ ID NO: 5. In another embodiment, the ActA fragment is any other ActA fragment known in the art.

In another embodiment, the recombinant nucleotide encoding a fragment of an

ActA protein comprises the sequence set forth in SEQ ID NO: 6:

Atgcgtgcgatgatggtggttttcattactgccaattgcattacgattaa
ccccgacataatatttgcagcgacagatagcgaagattctagtctaaaca
cagatgaatgggaagaagaaaaaacagaagagcaaccaagcgaggtaaat
acgggaccaagatacgaaactgcacgtgaagtaagttcacgtgatattaa
agaactagaaaaatcgaataaagtgagaaatacgaacaaagcagacctaa
tagcaatgttgaaagaaaaagcagaaaaaggtccaaatatcaataataac
aacagtgaacaaactgagaatgcggctataaatgaagaggcttcaggagc
cgaccgaccagctatacaagtggagcgtcgtcatccaggattgccatcgg
atagcgcagcggaaattaaaaaaagaaggaaagccatagcatcatcggat
agtgagcttgaaagccttacttatccggataaaccaacaaaagtaaataa
gaaaaaagtggcgaaagagtcagttgcggatgcttctgaaagtgacttag
attctagcatgcagtcagcagatgagtcttcaccacaacctttaaaagca
aaccaacaaccatttttccctaaagtatttaaaaaaataaaagatgcggg
gaaatgggtacgtgataaaatcgacgaaaatcctgaagtaaagaaagcga
ttgttgataaaagtgcagggttaattgaccaattattaaccaaaaagaaa
agtgaagaggtaaatgcttcggacttcccgccaccacctacggatgaaga
gttaagacttgctttgccagagacaccaatgcttcttggttttaatgctc
ctgctacatcagaaccgagctcattcgaatttccaccaccacctacggat
gaagagttaagacttgctttgccagagacgccaatgcttcttggttttaa
tgctcctgctacatcggaaccgagctcgttcgaatttccaccgcctccaa
cagaagatgaactagaaatcatccgggaaacagcatcctcgctagattct
agttttacaagaggggatttagctagtttgagaaatgctattaatcgcca
tagtcaaaatttctctgatttcccaccaatcccaacagaagaagagttga
acgggagaggcggtagacca.

In another embodiment, the recombinant nucleotide has the sequence set forth in SEQ ID NO: 6. In another embodiment, the recombinant nucleotide comprises any other sequence that encodes a fragment of an ActA protein.

In another embodiment of the methods and compositions of the present invention, a PEST amino acid AA sequence is fused to the E7 or E6 antigen. As disclosed herein, recombinant Listeria strains expressing PEST amino acid sequence-antigen fusions induce anti-tumor immunity (Example 3) and generate antigen-specific, tumor-infiltrating T cells (Example 4). Further, enhanced cell mediated immunity was demonstrated for fusion proteins comprising an antigen and LLO containing the PEST amino acid AA sequence

(SEQ ID NO: 1)

KENSISSMAPPASPPASPKTPIEKKHADEIDK.

Thus, fusion of an antigen to other LM PEST amino acid sequences and PEST amino acid sequences derived from other prokaryotic organisms will also enhance immunogenicity of the antigen. The PEST amino acid AA sequence has, in another embodiment, a sequence selected from SEQ ID NO: 7-12. In another embodiment, the PEST amino acid sequence is a PEST amino acid sequence from the LM ActA protein. In another embodiment, the PEST amino acid sequence is KTEEQPSEVNTGPR (SEQ ID NO: 7), KASVTDTSEGDLDSSMQSADESTPQPLK (SEQ ID NO: 8), KNEEVNASDFPPPPTDEELR (SEQ ID NO: 9), or RGGIPTSEEFSSLNSGDFTDDENSETTEEEIDR (SEQ ID NO: 10). In another embodiment, the PEST amino acid sequence is from Streptolysin O protein of Streptococcus sp. In another embodiment, the PEST amino acid sequence is from Streptococcus pyogenes Streptolysin O, e.g. KQNTASTETTTTNEQPK (SEQ ID NO: 11) at AA 35-51. In another embodiment, the PEST amino acid sequence is from Streptococcus equisimilis Streptolysin O, e.g. KQNTANTETTTTNEQPK (SEQ ID NO:12) at AA 38-54. In another embodiment, the PEST amino acid sequence is another PEST amino acid AA sequence derived from a prokaryotic organism. In another embodiment, the PEST amino acid sequence is any other PEST amino acid sequence known in the art.

PEST amino acid sequences of other prokaryotic organism can be identified in accordance with methods such as described by, for example Rechsteiner and Rogers (1996, Trends Biochem. Sci. 21:267-271) for LM. Alternatively, PEST amino acid AA sequences from other prokaryotic organisms can also be identified based by this method. Other prokaryotic organisms wherein PEST amino acid AA sequences would be expected to include, but are not limited to, other Listeria species. In another embodiment, the PEST amino acid sequence is embedded within the antigenic protein. Thus, in another embodiment, “fusion” refers to an antigenic protein comprising both the antigen and the PEST amino acid amino acid sequence either linked at one end of the antigen or embedded within the antigen.

In another embodiment, the PEST amino acid sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each 30-35 AA stretch by assigning a value of 1 to the amino acids Ser, Thr, Pro, Glu, Asp, Asn, or Gln. The coefficient value (CV) for each of the PEST residue is 1 and for each of the other AA (non-PEST) is 0.

In another embodiment, the LLO protein, ActA protein, or fragment thereof of the present invention need not be that which is set forth exactly in the sequences set forth herein, but rather other alterations, modifications, or changes can be made that retain the functional characteristics of an LLO or ActA protein fused to an antigen as set forth elsewhere herein. In another embodiment, the present invention utilizes an analog of an LLO protein, ActA protein, or fragment thereof. Analogs differ, in another embodiment, from naturally occurring proteins or peptides by conservative AA sequence differences or by modifications which do not affect sequence, or by both.

In another embodiment, either a whole E7 protein or a fragment thereof is fused to a LLO protein, ActA protein, or PEST amino acid sequence-containing peptide to generate a recombinant peptide of methods of the present invention. The E7 protein that is utilized (either whole or as the source of the fragments) has, in another embodiment, the sequence

(SEQ ID No: 13)

MHGDTPTLHEYMLDLQPETTDLYCYEQLNDSSEEEDEIDGPAGQAEPD
RAHYNIVTFCCKCDSTLRLCVQSTHVDIRTLEDLLMGTLGIVCPICSQK
P.

In another embodiment, the E7 protein is a homologue of SEQ ID No: 13. In another embodiment, the E7 protein is a variant of SEQ ID No: 13. In another embodiment, the E7 protein is an isomer of SEQ ID No: 13. In another embodiment, the E7 protein is a fragment of SEQ ID No: 13. In another embodiment, the E7 protein is a fragment of a homologue of SEQ ID No: 13. In another embodiment, the E7 protein is a fragment of a variant of SEQ ID No: 13. In another embodiment, the E7 protein is a fragment of an isomer of SEQ ID No: 13.

In another embodiment, the sequence of the E7 protein is:

(SEQ ID No: 14)

MHGPKATLQDIVLHLEPQNEIPVDLLCHEQLSDSEEENDEIDGVNHQHLP
ARRAEPQRHTMLCMCCKCEARIELVVESSADDLRAFQQLFLNTLSFVCPW
CASQQ.

In another embodiment, the E7 protein is a homologue of SEQ ID No: 14. In another embodiment, the E7 protein is a variant of SEQ ID No: 14. In another embodiment, the E7 protein is an isomer of SEQ ID No: 14. In another embodiment, the E7 protein is a fragment of SEQ ID No: 14. In another embodiment, the E7 protein is a fragment of a homologue of SEQ ID No: 14. In another embodiment, the E7 protein is a fragment of a variant of SEQ ID No: 14. In another embodiment, the E7 protein is a fragment of an isomer of SEQ ID No: 14.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries.

In another embodiment, either a whole E6 protein or a fragment thereof is fused to a LLO protein, ActA protein, or PEST amino acid sequence-containing peptide to generate a recombinant peptide of methods of the present invention. The E6 protein that is utilized (either whole or as the source of the fragments) has, in another embodiment, the sequence

(SEQ ID No: 15)

MHQKRTAMFQDPQERPRKLPQLCTELQTTIHDIILECVYCKQQLLRREVY
DFAFRDLCIVYRDGNPYAVCDKCLKFYSKISEYRHYCYSLYGTTLEQQYN
KPLCDLLIRCINCQKPLCPEEKQRHLDKKQRFHNIRGRWTGRCMSCCRSS
RTRRETQL.

In another embodiment, the E6 protein is a homologue of SEQ ID No: 15. In another embodiment, the E6 protein is a variant of SEQ ID No: 15. In another embodiment, the E6 protein is an isomer of SEQ ID No: 15. In another embodiment, the E6 protein is a fragment of SEQ ID No: 15. In another embodiment, the E6 protein is a fragment of a homologue of SEQ ID No: 15. In another embodiment, the E6 protein is a fragment of a variant of SEQ ID No: 15. In another embodiment, the E6 protein is a fragment of an isomer of SEQ ID No: 15.

In another embodiment, the sequence of the E6 protein is:

(SEQ ID No: 16)

MARFEDPTRRPYKLPDLCTELNTSLQDIEITCVYCKTVLELTEVFEFAFK
DLFVVYRDSIPHAACHKCIDFYSRIRELRHYSDSVYGDTLEKLTNTGLYN
LLIRCLRCQKPLNPAEKLRHLNEKRRFHNIAGHYRGQCHSCCNRARQERL
QRRRETQV.

In another embodiment, the E6 protein is a homologue of SEQ ID No: 16. In another embodiment, the E6 protein is a variant of SEQ ID No: 16. In another embodiment, the E6 protein is an isomer of SEQ ID No: 16. In another embodiment, the E6 protein is a fragment of SEQ ID No: 16. In another embodiment, the E6 protein is a fragment of a homologue of SEQ ID No: 16. In another embodiment, the E6 protein is a fragment of a variant of SEQ ID No: 16. In another embodiment, the E6 protein is a fragment of an isomer of SEQ ID No: 16. In another embodiment, the E6 protein has a sequence set forth in one of the following GenBank entries: M24215, M14119, NC_004500, V01116, X62843, or M14119. In another embodiment, the E6 protein is a homologue of a sequence from one of the above GenBank entries. In another embodiment, the E6 protein is a variant of a sequence from one of the above GenBank entries. In another embodiment, the E6 protein is an isomer of a sequence from one of the above GenBank entries. In another embodiment, the E6 protein is a fragment of a sequence from one of the above GenBank entries. In another embodiment, the E6 protein is a fragment of a homologue of a sequence from one of the above GenBank entries. In another embodiment, the E6 protein is a fragment of a variant of a sequence from one of the above GenBank entries. In another embodiment, the E6 protein is a fragment of an isomer of a sequence from one of the above GenBank entries.

In another embodiment, “homology” refers to identity to an LLO sequence (e.g. to one of SEQ ID No: 2-4) of greater than 70%. In another embodiment, “homology” refers to identity to one of SEQ ID No: 2-4 of greater than 64%. In another embodiment, “homology” refers to identity to one of SEQ ID No: 2-4 of greater than 68%. In another embodiment, “homology” refers to identity to one of SEQ ID No: 2-4 of greater than 72%. In another embodiment, “homology” refers to identity to one of SEQ ID No: 2-4 of greater than 75%. In another embodiment, “homology” refers to identity to one of SEQ ID No: 2-4 of greater than 78%. In another embodiment, “homology” refers to identity to one of SEQ ID No: 2-4 of greater than 80%. In another embodiment, “homology” refers to identity to one of SEQ ID No: 2-4 of greater than 82%. In another embodiment, “homology” refers to identity to one of SEQ ID No: 2-4 of greater than 83%. In another embodiment, “homology” refers to identity to one of SEQ ID No: 2-4 of greater than 85%. In another embodiment, “homology” refers to identity to one of SEQ ID No: 2-4 of greater than 87%. In another embodiment, “homology” refers to identity to one of SEQ ID No: 2-4 of greater than 88%. In another embodiment, “homology” refers to identity to one of SEQ ID No: 2-4 of greater than 90%. In another embodiment, “homology” refers to identity to one of SEQ ID No: 2-4 of greater than 92%. In another embodiment, “homology” refers to identity to one of SEQ ID No: 2-4 of greater than 93%. In another embodiment, “homology” refers to identity to one of SEQ ID No: 2-4 of greater than 95%. In another embodiment, “homology” refers to identity to one of SEQ ID No: 2-4 of greater than 96%. In another embodiment, “homology” refers to identity to one of SEQ ID No: 2-4 of greater than 97%. In another embodiment, “homology” refers to identity to one of SEQ ID No: 2-4 of greater than 98%. In another embodiment, “homology” refers to identity to one of SEQ ID No: 2-4 of greater than 99%. In another embodiment, “homology” refers to identity to one of SEQ ID No: 2-4 of 100%.

In another embodiment, “homology” refers to identity to an E7 sequence (e.g. to one of SEQ ID No: 13-14) of greater than 70%. In another embodiment, “homology” refers to identity to one of SEQ ID No: 13-14 of greater than 62%. In another embodiment, “homology” refers to identity to one of SEQ ID No: 13-14 of greater than 64%. In another embodiment, “homology” refers to identity to one of SEQ ID No: 13-14 of greater than 68%. In another embodiment, “homology” refers to identity to one of SEQ ID No: 13-14 of greater than 72%. In another embodiment, “homology” refers to identity to one of SEQ ID No: 13-14 of greater than 75%. In another embodiment, “homology” refers to identity to one of SEQ ID No: 13-14 of greater than 78%. In another embodiment, “homology” refers to identity to one of SEQ ID No: 13-14 of greater than 80%. In another embodiment, “homology” refers to identity to one of SEQ ID No: 13-14 of greater than 82%. In another embodiment, “homology” refers to identity to one of SEQ ID No: 13-14 of greater than 83%. In another embodiment, “homology” refers to identity to one of SEQ ID No: 13-14 of greater than 85%. In another embodiment, “homology” refers to identity to one of SEQ ID No: 13-14 of greater than 87%. In another embodiment, “homology” refers to identity to one of SEQ ID No: 13-14 of greater than 88%. In another embodiment, “homology” refers to identity to one of SEQ ID No: 13-14 of greater than 90%. In another embodiment, “homology” refers to identity to one of SEQ ID No: 13-14 of greater than 92%. In another embodiment, “homology” refers to identity to one of SEQ ID No: 13-14 of greater than 93%. In another embodiment, “homology” refers to identity to one of SEQ ID No: 13-14 of greater than 95%. In another embodiment, “homology” refers to identity to one of SEQ ID No: 13-14 of greater than 96%. In another embodiment, “homology” refers to identity to one of SEQ ID No: 13-14 of greater than 97%. In another embodiment, “homology” refers to identity to one of SEQ ID No: 13-14 of greater than 98%. In another embodiment, “homology” refers to identity to one of SEQ ID No: 13-14 of greater than 99%. In another embodiment, “homology” refers to identity to one of SEQ ID No: 13-14 of 100%.

In another embodiment, “homology” refers to identity to an E6 sequence (e.g. to one of SEQ ID No: 15-16) of greater than 70%. In another embodiment, “homology” refers to identity to one of SEQ ID No: 15-16 of greater than 64%. In another embodiment, “homology” refers to identity to one of SEQ ID No: 15-16 of greater than 68%. In another embodiment, “homology” refers to identity to one of SEQ ID No: 15-16 of greater than 72%. In another embodiment, “homology” refers to identity to one of SEQ ID No: 15-16 of greater than 75%. In another embodiment, “homology” refers to identity to one of SEQ ID No: 15-16 of greater than 78%. In another embodiment, “homology” refers to identity to one of SEQ ID No: 15-16 of greater than 80%. In another embodiment, “homology” refers to identity to one of SEQ ID No: 15-16 of greater than 82%. In another embodiment, “homology” refers to identity to one of SEQ ID No: 15-16 of greater than 83%. In another embodiment, “homology” refers to identity to one of SEQ ID No: 15-16 of greater than 85%. In another embodiment, “homology” refers to identity to one of SEQ ID No: 15-16 of greater than 87%. In another embodiment, “homology” refers to identity to one of SEQ ID No: 15-16 of greater than 88%. In another embodiment, “homology” refers to identity to one of SEQ ID No: 15-16 of greater than 90%. In another embodiment, “homology” refers to identity to one of SEQ ID No: 15-16 of greater than 92%. In another embodiment, “homology” refers to identity to one of SEQ ID No: 15-16 of greater than 93%. In another embodiment, “homology” refers to identity to one of SEQ ID No: 15-16 of greater than 95%. In another embodiment, “homology” refers to identity to one of SEQ ID No: 15-16 of greater than 96%. In another embodiment, “homology” refers to identity to one of SEQ ID No: 15-16 of greater than 97%. In another embodiment, “homology” refers to identity to one of SEQ ID No: 15-16 of greater than 98%. In another embodiment, “homology” refers to identity to one of SEQ ID No: 15-16 of greater than 99%. In another embodiment, “homology” refers to identity to one of SEQ ID No: 15-16 of 100%.

In another embodiment, “homology” refers to identity to a PEST amino acid sequence (e.g. to one of SEQ ID No: 1, and 7-12) or to an ActA sequence (e.g. to one of SEQ ID No: 5-6) of greater than 70%. In another embodiment, “homology” refers to identity to one of SEQ ID No: 1, and 7-12 or SEQ ID No: 5-6 of greater than 60%. In another embodiment, “homology” refers to identity to one of SEQ ID No: 1, and 7-12 or SEQ ID No: 5-6 of greater than 64%. In another embodiment, “homology” refers to identity to one of SEQ ID No: 1, and 7-12 or SEQ ID No: 5-6 of greater than 68%. In another embodiment, “homology” refers to identity to one of SEQ ID No: 1, and 7-12 or SEQ ID No: 5-6 of greater than 72%. In another embodiment, “homology” refers to identity to one of SEQ ID No: 1, and 7-12 or SEQ ID No: 5-6 of greater than 75%. In another embodiment, “homology” refers to identity to one of SEQ ID No: 1, and 7-12 or SEQ ID No: 5-6 of greater than 78%. In another embodiment, “homology” refers to identity to one of SEQ ID No: 1, and 7-12 or SEQ ID No: 5-6 of greater than 80%. In another embodiment, “homology” refers to identity to one of SEQ ID No: 1, and 7-12 or SEQ ID No: 5-6 of greater than 82%. In another embodiment, “homology” refers to identity to one of SEQ ID No: 1, and 7-12 or SEQ ID No: 5-6 of greater than 83%. In another embodiment, “homology” refers to identity to one of SEQ ID No: 1, and 7-12 or SEQ ID No: 5-6 of greater than 85%. In another embodiment, “homology” refers to identity to one of SEQ ID No: 1, and 7-12 or SEQ ID No: 5-6 of greater than 87%. In another embodiment, “homology” refers to identity to one of SEQ ID No: 1, and 7-12 or SEQ ID No: 5-6 of greater than 88%. In another embodiment, “homology” refers to identity to one of SEQ ID No: 1, and 7-12 or SEQ ID No: 5-6 of greater than 90%. In another embodiment, “homology” refers to identity to one of SEQ ID No: 1, and 7-12 or SEQ ID No: 5-6 of greater than 92%. In another embodiment, “homology” refers to identity to one of SEQ ID No: 1, and 7-12 or SEQ ID No: 5-6 of greater than 93%. In another embodiment, “homology” refers to identity to one of SEQ ID No: 1, and 7-12 or SEQ ID No: 5-6 of greater than 95%. In another embodiment, “homology” refers to identity to one of SEQ ID No: 1, and 7-12 or SEQ ID No: 5-6 of greater than 96%. In another embodiment, “homology” refers to identity to one of SEQ ID No: 1, and 7-12 or SEQ ID No: 5-6 of greater than 97%. In another embodiment, “homology” refers to identity to one of SEQ ID No: 1, and 7-12 or SEQ ID No: 5-6 of greater than 98%. In another embodiment, “homology” refers to identity to one of SEQ ID No: 1, and 7-12 or SEQ ID No: 5-6 of greater than 99%. In another embodiment, “homology” refers to identity to one of SEQ ID No: 1, and 7-12 or SEQ ID No: 5-6 of 100%.

Protein and/or peptide homology for any AA sequence listed herein is determined, in one embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ, in other embodiments, the use of the Smith and Waterman algorithms, and/or global/local or BLOCKS alignments for analysis, for example.

In another embodiment, the LLO protein, ActA protein, or fragment thereof is attached to the antigen by chemical conjugation. In another embodiment, glutaraldehyde is used for the conjugation. In another embodiment, the conjugation is performed using any suitable method known in the art.

In another embodiment, fusion proteins of the present invention are prepared by any suitable method, including, for example, cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid.

In another embodiment, the LLO protein, ActA protein, or fragment thereof and the antigen, or fragment thereof are conjugated by a means known to those of skill in the art. In another embodiment, the antigen, or fragment thereof is conjugated, either directly or through a linker (spacer), to the ActA protein or LLO protein. In another embodiment, the chimeric molecule is recombinantly expressed as a single-chain fusion protein.

In another embodiment, a fusion peptide of the present invention is synthesized using standard chemical peptide synthesis techniques. In another embodiment, the chimeric molecule is synthesized as a single contiguous polypeptide. In another embodiment, the LLO protein, ActA protein, or fragment thereof; and the antigen, or fragment thereof are synthesized separately, then fused by condensation of the amino terminus of one molecule with the carboxyl terminus of the other molecule, thereby forming a peptide bond. In another embodiment, the ActA protein or LLO protein and antigen are each condensed with one end of a peptide spacer molecule, thereby forming a contiguous fusion protein.

In another embodiment, the peptides and proteins of the present invention are prepared by solid-phase peptide synthesis (SPPS) as described by Stewart et al. in Solid Phase Peptide Synthesis, 2nd Edition, 1984, Pierce Chemical Company, Rockford, Ill.; or as described by Bodanszky and Bodanszky (The Practice of Peptide Synthesis, 1984, Springer-Verlag, New York). In another embodiment, a suitably protected AA residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial AA, and couple thereto of the carboxyl end of the next AA in the sequence of the desired peptide. This AA is also suitably protected. The carboxyl of the incoming AA can be activated to react with the N-terminus of the support-bound AA by formation into a reactive group such as formation into a carbodiimide, a symmetric acid anhydride or an “active ester” group such as hydroxybenzotriazole or pentafluorophenly esters.

In another embodiment, the present invention provides a kit comprising vaccine of the present invention, an applicator, and instructional material that describes use of the methods of the invention. Although model kits are described below, the contents of other useful kits will be apparent to the skilled artisan in light of the present disclosure. Each of these kits represents a separate embodiment of the present invention.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, pets mice and humans. The subject may also include livestock. In one embodiment, the term “subject” does not exclude an individual that is healthy in all respects and does not have or show signs of disease or disorder.

Specific embodiments include:
1. An immunogenic composition comprising a recombinant Listeria strain comprising a recombinant nucleic acid, said nucleic acid comprising a first open reading frame encoding a recombinant polypeptide comprising an N-terminal fragment of an LLO protein operably linked or fused to at least one heterologous antigen or fragment thereof, wherein said recombinant nucleic acid further comprises a second open reading frame encoding a metabolic enzyme.
2. The immunogenic composition of embodiment 1, wherein said N-terminal fragment of an LLO protein comprises SEQ ID NO: 2.
3. The immunogenic composition of embodiments 1-2, wherein said recombinant Listeria strain is a recombinant Listeria monocytogenes strain.
4. The immunogenic composition of embodiments 1-3, wherein, wherein said recombinant polypeptide is expressed by said recombinant Listeria strain.
5. The immunogenic composition of embodiments 1-4, wherein said nucleic acid is in an extrachromosomal plasmid in said recombinant Listeria strain.
6. The immunogenic composition of embodiments 1-5, wherein said plasmid is stably maintained in said recombinant Listeria strain.
7. The immunogenic composition of embodiments 1-6, wherein said Listeria strain comprises a mutation, deletion or inactivation in the genomic dal, dat, and actA genes.
8. The immunogenic composition of embodiments 1-7, wherein said metabolic enzyme complements said mutation, deletion or inactivation.
9. The immunogenic composition of embodiments 1-8, wherein said first open reading frame encodes two to four heterologous antigens or functional fragments operably linked in tandem, wherein the most N-terminus heterologous antigen or fragment thereof is operably linked or fused to an N-terminal LLO protein fragment at the N-terminus.
10. The immunogenic composition of embodiments 1-9, wherein said first open reading frame encodes a recombinant polypeptide comprising four heterologous antigens or functional fragments operably linked in tandem, wherein said operably linked heterologous antigens are fused to an N-terminal LLO protein fragment at the N-terminus of the most N-terminus heterologous antigen.
11. The immunogenic composition of embodiment 10, wherein a structure of said recombinant polypeptide encoded by said first open reading frame comprises an N-terminal LLO-antigen 1-antigen 2-antigen 3-antigen 4.
12. The immunogenic composition of any one of embodiments 10-11, wherein said two to four heterologous antigens or four heterologous antigens are selected from the group consisting of HPV16 E6, HPV16 E7, HPV18 E6, and HPV18 E7 antigens.
13. The immunogenic composition of any one of embodiments 10-12, wherein antigen 1 is HPV16 E6, antigen 2 is HPV16 E7, antigen3 is HPV18 E6 and antigen 4 is HPV18 E7.
14. The immunogenic composition of any one of embodiments 10-13, wherein said first open reading frame further encodes a protein tag sequence operably linked at the C-terminus of the last heterologous antigen.
15. The immunogenic composition of embodiment 14, wherein said protein tag sequence is SIINFEKL-6xHIS sequence.
16. The immunogenic composition of any one of embodiments 14-15, wherein said first open reading frame comprises SEQ ID NO: 32.
17. The immunogenic composition of any one of embodiments 14-15, wherein said first open reading frame encodes a protein comprising SEQ ID NO: 33.
18. The immunogenic composition of any one of embodiments 1-17, wherein said recombinant Listeria strain has been passaged through an animal host.
19. The immunogenic composition of any one of embodiments 1-18, further comprising an adjuvant.
20. The immunogenic composition of embodiment 19, wherein said adjuvant is selected from the list comprising a GM-CSF protein, saponin QS21, monophosphoryl lipid A, SBAS2, an unmethylated CpG-containing oligonucleotide, an immune-stimulating cytokine, a quill glycoside, a bacterial toxin, and a bacterial mitogen.
21. The immunogenic composition of any one of embodiments 1-20, wherein said recombinant Listeria strain has been stored in a frozen or lyophilized cell bank.
22. A method of treating tumor or cancer in a subject, the method comprising the step of administering to said subject an immunogenic composition of embodiments 1-21.
23. A method of protecting a human subject against a tumor or cancer, comprising the step of administering to said subject the composition of embodiments 1-21.
24. A method for inducing an anti-tumor cytotoxic T cell response in a human subject, comprising the step of administering to said subject a composition comprising a recombinant Listeria strain of embodiments 1-21.

The following examples are presented in order to more fully illustrate the preferred embodiments of the invention. They should in no way be construed, however, as limiting the broad scope of the invention.

### Experimental Details Section

### EXAMPLE 1

### LLO-Antigen Fusions Induce Anti-Tumor Immunity

### Materials And Experimental Methods (Examples 1-2)

### Cell Lines

The C57BL/6 syngeneic TC-1 tumor was immortalized with HPV-16 E6 and E7 and transformed with the c-Ha-ras oncogene. TC-1, disclosed by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/m1 G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

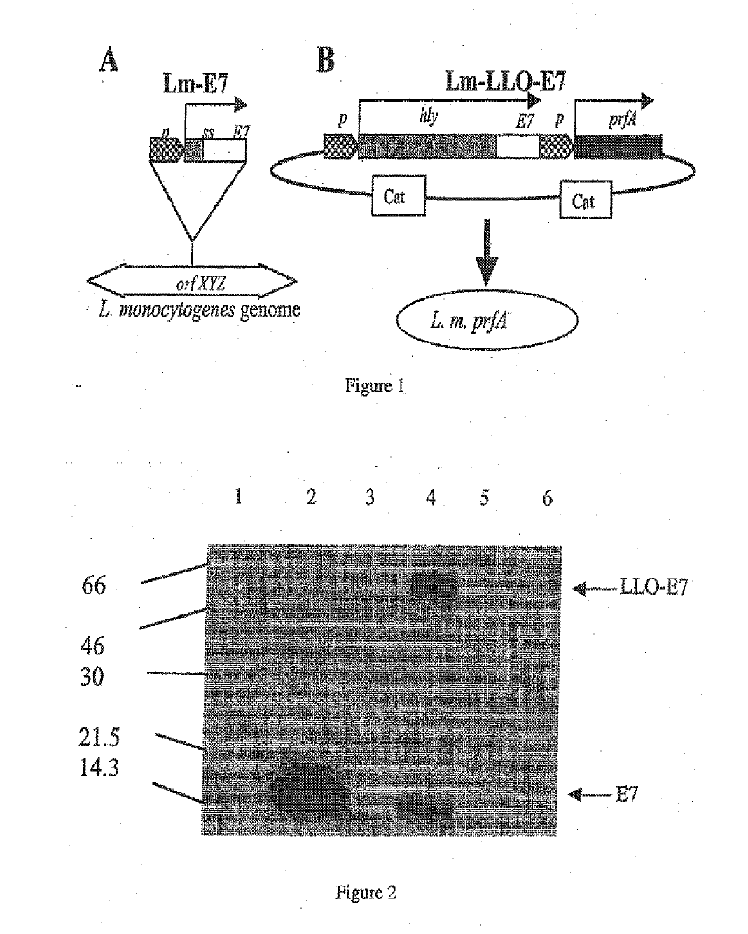

Listeria strains used were Lm-LLO-E7 (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 17; XhoI site is underlined) and 5′-GGGGACTAGTTTATGGTTTCTGAGAACA-3′ (SEQ ID No: 18; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor PrfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, referred to below as “ΔLLO”), which is joined by the Xhol site to the E7 gene, yielding a hly-E7 fusion gene that is transcribed and secreted as LLO-E7. Transformation of a prfA negative strain of Listeria, XFL-7 (disclosed by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 19; NheI site is underlined) and 5′-CTCCCTCGAGATCATAATTTACTTCATC-3′ (SEQ ID No: 20; XhoI site is underlined). The prfA gene was PCR amplified using primers 5′-GACTACAAGGACGATGACCGACAAGTGATAACCCGGGATCTAAATAAATCCGTT T-3′ (SEQ ID No: 27; XbaI site is underlined) and 5′-CCCGTCGACCAGCTCTTCTTGGTGAAG-3′ (SEQ ID No: 21; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 22; BamHI site is underlined) and 5′-GCTCTAGATTATGGTTTCTGAG-3′ (SEQ ID No: 23; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

### Western Blotting

Listeria strains were grown in Luria-Bertoni medium at 37° C. and were harvested at the same optical density measured at 600 nm. The supernatants were TCA precipitated and resuspended in lx sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

### Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

### ⁵¹Cr Release Assay

C57BL/6 mice, 6-8 wk old, were immunized i.p. with 0.1LD₅₀ Lm-LLO-E7,

Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)−spontaneous cpm)/(total cpm−spontaneous cpm)]×100.

### TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm−no Ag cpm.

### Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were disclosed by Dr. Larry R. Pease (Mayo Clinic, Rochester, Minn.) and by the NIAID Tetramer Core Facility and the NIH AIDS Research and Reference Reagent Program. Tetramer⁺, CD8⁺, CD62L^{low} cells were analyzed.

### B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

### Statistics

For comparisons of tumor diameters, mean and SD of tumor size for each group were determined, and statistical significance was determined by Student's t test. p≤0.05 was considered significant.

### Results

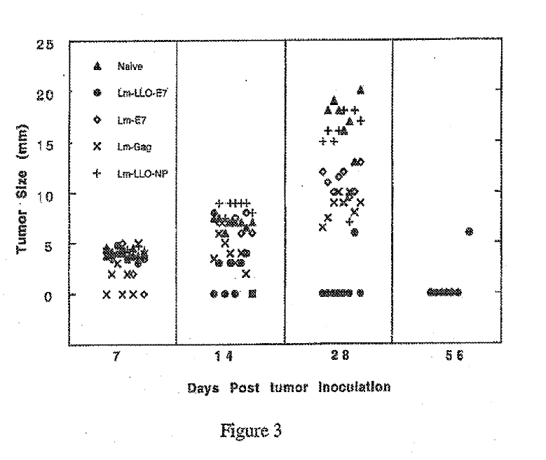

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ΔLLO enhances the immunogenicity of the antigen.

### EXAMPLE 2

### LM-LLO-E7 Treatment Elicits Tc-1 Specific Splenocyte Proliferation

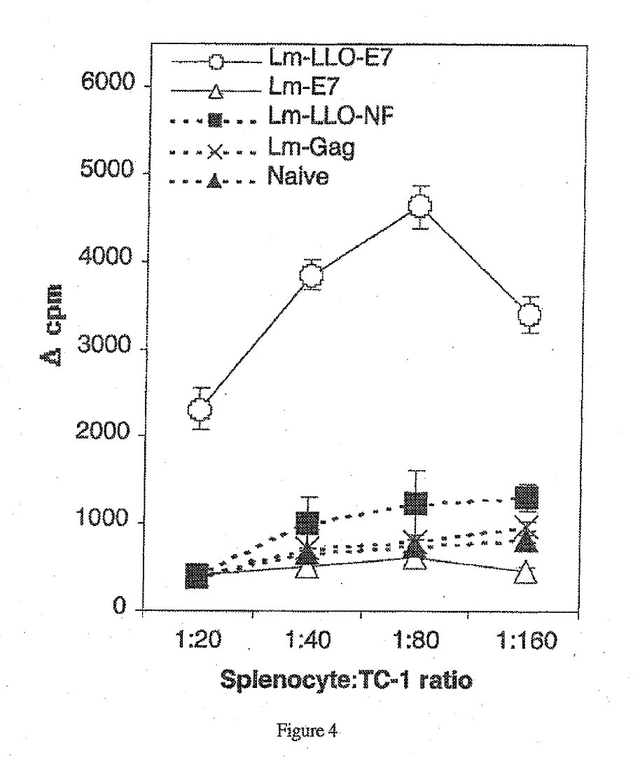

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### EXAMPLE 3

### Fusion of E7 to LLO, ActA, or a Pest Amino Acid Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4 ° C. overnight in tubes containing 2 milliliters (ml) of RP 10 medium on ice. Tumors were minced with forceps, cut into 2 mm blocks, and incubated at 37 ° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4 ° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low} ) CD8⁺ T cells were calculated.

For tetramer staining, H-2D^{b} tetramer was loaded with phycoerythrin (PE)-conjugated E7 peptide (RAHYNIVTF, SEQ ID NO: 24), stained at rt for 1 hour, and stained with anti-allophycocyanin (APC) conjugated MEL-14 (CD62L) and FITC-conjugated CD8□ at 4° C. for 30 min. Cells were analyzed comparing tetramer⁺CD8⁺ CD62L^{low} cells in the spleen and in the tumor.

### Results

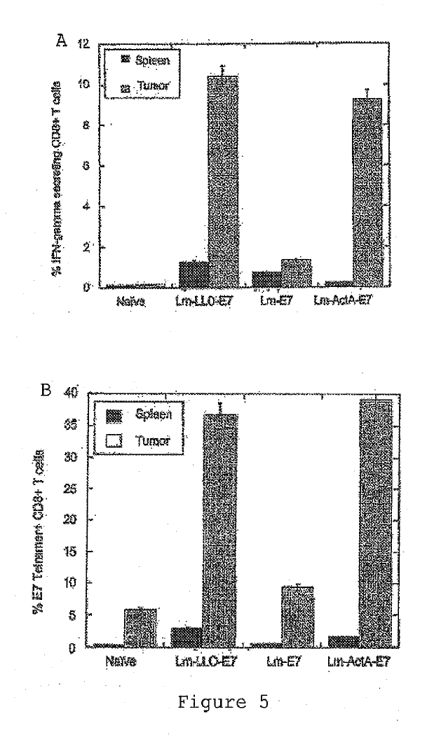

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 5A) and tetramer-specific CD8⁺ cells (FIG. 5B) than in Lm-E7 or naive mice.

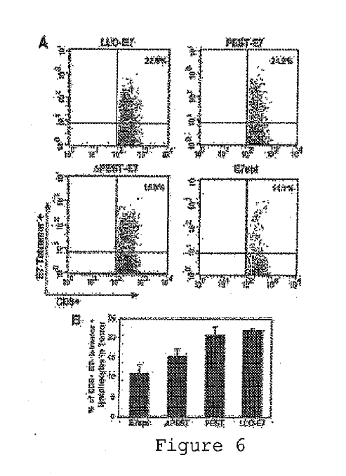

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 6A). This result was reproducible over three experiments (FIG. 6B).

Thus, Lm-LLO-E7, Lm-ActA-E7, and Lm-PEST-E7 are each efficacious at induction of tumor-infiltrating CD8⁺ T cells and tumor regression.

### EXAMPLE 4

### Passaging of Listeria Vaccine Vectors Through Mice Elicits Increased Immune Responses to Heterologous and Endogenous Antigens

### Materials and Experimental Methods

### Bacterial Strains

L. monocytogenes strain 10403S, serotype 1 (ATCC, Manassas, Va.) was the wild type organism used in these studies and the parental strain of the constructs described below. Strain 10403S has an LD₅₀ of approximately 5×10⁴ CFU when injected intraperitoneally into BALB/c mice. “Lm-Gag” is a recombinant LM strain containing a copy of the HIV-1 strain HXB (subtype B laboratory strain with a syncytia-forming phenotype) gag gene stably integrated into the Listerial chromosome using a modified shuttle vector pKSV7. Gag protein was expressed and secreted by the strain, as determined by Western blot. All strains were grown in brain-heart infusion (BHI) broth or agar plates (Difco Labs, Detroit, Mich.).

### Bacterial Culture

Bacteria from a single clone expressing the passenger antigen and/or fusion protein were selected and cultured in BHI broth overnight. Aliquots of this culture were frozen at ⁻70° C. with no additives. From this stock, cultures were grown to 0.1-0.2 O.D. at 600 nm, and aliquots were again frozen at −70° C. with no additives. To prepare cloned bacterial pools, the above procedure was used, but after each passage a number of bacterial clones were selected and checked for expression of the target antigen, as described herein. Clones in which expression of the foreign antigen was confirmed were used for the next passage.

### Passage of Bacteria in Mice

6-8 week old female BALB/c (H-2d) mice were purchased from Jackson Laboratories (Bar Harbor, Me.) and were maintained in a pathogen-free microisolator environment. The titer of viable bacteria in an aliquot of stock culture, stored frozen at −70° C., was determined by plating on BHI agar plates on thawing and prior to use. In all, 5×10⁵ bacteria were injected intravenously into BALB/c mice. After 3 days, spleens were harvested, homogenized, and serial dilutions of the spleen homogenate were incubated in BHI broth overnight and plated on BHI agar plates. For further passage, aliquots were again grown to 0.1-0.2 O.D., frozen at −70 ° C., and bacterial titer was again determined by serial dilution. After the initial passage (passage 0), this sequence was repeated for a total of 4 times.

### Intracellular Cytokine Stain for IFN-Gamma

Lymphocytes were cultured for 5 hours in complete RPMI-10 medium supplemented with 50 U/ml human recombinant IL-2 and 1 microliter/ml Brefeldin A (Golgistop™; PharMingen, San Diego, Calif.) in the presence or absence of either the cytotoxic T-cell (CTL) epitope for HIV-GAG (AMQMLKETI; SEQ ID No: 25), Listeria LLO (GYKDGNEYI; SEQ ID No: 26) or the HPV virus gene E7 (RAHYNIVTF (SEQ ID No: 24), at a concentration of 1 micromole. Cells were first surface-stained, then washed and subjected to intracellular cytokine stain using the Cytofix/Cytoperm kit in accordance with the manufacturer's recommendations (PharMingen, San Diego, Calif.). For intracellular IFN-gamma stain, FITC-conjugated rat anti-mouse IFN-gamma monoclonal antibody (clone XMG 1.2) and its isotype control Ab (rat IgG1; both from PharMingen) was used. In all, 10⁶ cells were stained in PBS containing 1% Bovine Serum Albumin and 0.02% sodium azide (FACS Buffer) for 30 minutes at 4° C. followed by 3 washes in FACS buffer. Sample data were acquired on either a FACScan™ flowcytometer or FACSCalibur™ instrument (Becton Dickinson, San Jose, Calif.). Three-color flow cytometry for CD8 (PERCP conjugated, rat anti-mouse, clone 53-6.7 Pharmingen, San Diego, Calif.), CD62L (APC conjugated, rat anti-mouse, clone MEL-14), and intracellular IFN-gamma was performed using a FACSCalibur™ flow cytometer, and data were further analyzed with CELLQuest software (Becton Dickinson, Mountain View, Calif.). Cells were gated on CD8 high and CD62L^{low} before they were analyzed for CD8⁺ and intracellular IFN-gamma staining.

### Results

Passaging in Mice Increases the Virulence of Recombinant Listeria monocytogenes

Three different constructs were used to determine the impact of passaging on recombinant Listeria vaccine vectors. Two of these constructs carry a genomic insertion of the passenger antigen: the first comprises the HIV gag gene (Lm-Gag), and the second comprises the HPV E7 gene (Lm-E7). The third (Lm-LLO-E7) comprises a plasmid with the fusion gene for the passenger antigen (HPV E7) fused with a truncated version of LLO and a gene encoding PrfA, the positive regulatory factor that controls Listeria virulence factors. This plasmid was used to complement a prfA negative mutant so that in a live host, selection pressures would favor conservation of the plasmid, because without it the bacterium is avirulent. All 3 constructs had been propagated extensively in vitro for many bacterial generations.

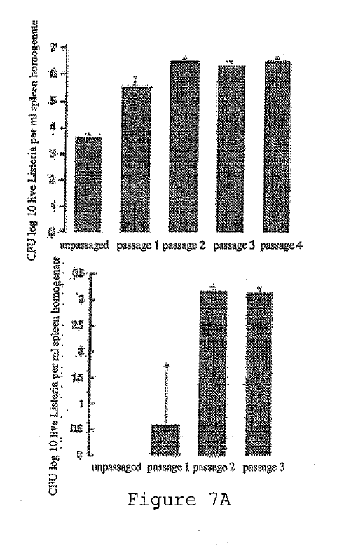

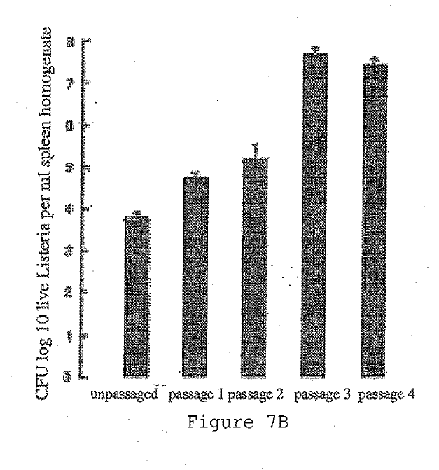

Passaging the bacteria resulted in an increase in bacterial virulence, as measured by numbers of surviving bacteria in the spleen, with each of the first 2 passages. For Lm-Gag and Lm-LLO-E7, virulence increased with each passage up to passage 2 (FIG. 7A). The plasmid-containing construct, Lm-LLO-E7, demonstrated the most dramatic increase in virulence. Prior to passage, the initial immunizing dose of Lm-LLO-E7 had to be increased to 10⁷ bacteria and the spleen had to be harvested on day 2 in order to recover bacteria (whereas an initial dose of 10⁵ bacteria for Lm-Gag was harvested on day 3). After the initial passage, the standard dosage of Lm-LLO-E7 was sufficient to allow harvesting on day 3. For Lm-E7, virulence increased by 1.5 orders of magnitude over unpassaged bacteria (FIG. 7B).

Thus, passage through mice increases the virulence of Listeria vaccine strains.

Passaging Increases the Ability of L. monocytogenes to Induce CD8⁺ T Cells

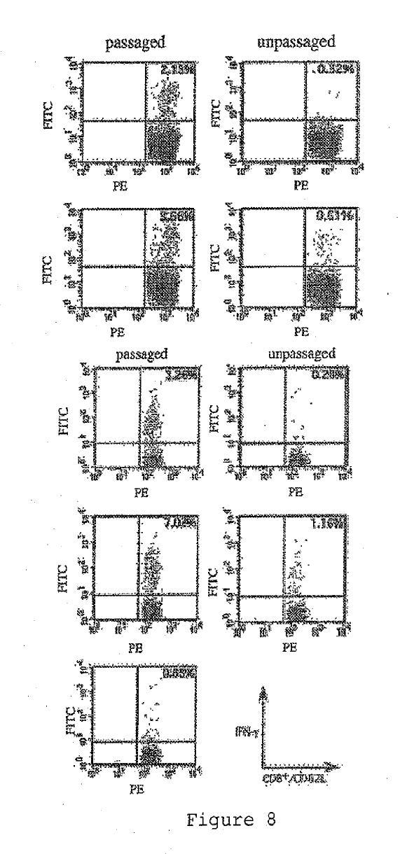

Next, the effect of passaging on induction of antigen-specific CD8⁺ T cells was determined by intracellular cytokine staining with immunodominant peptides specific for MHC-class I using HIV-Gag peptide AMQMLKETI (SEQ ID No: 25) and LLO 91-99 (GYKDGNEYI; SEQ ID No: 26). Injection of 10³ CFU passaged bacteria (Lm-Gag) into mice elicited significant numbers of HIV-Gag-specific CD8⁺ T cells, while the same dose of non-passaged Lm-Gag induced no detectable Gag-specific CD8⁺ T cells. Even increasing the dose of unpassaged bacteria 100-fold did not compensate for their relative avirulence; in fact, no detectable Gag-specific CD8⁺ T cells were elicited even at the higher dose. The same dose increase with passaged bacteria increased Gag-specific T cell induction by 50% (FIG. 8). The same pattern of induction of antigen-specific CD8⁺ T cells was observed with LLO-specific CD8⁺ T cells, showing that these results were not caused by the properties of the passenger antigen, since they were observed with LLO, an endogenous Listeria antigen.

Thus, passage through mice increases the immunogenicity of Listeria vaccine strains.

### EXAMPLE 5

### A Prfa-Containing Plasmid is Stable in an Lm Strain with a Prfa Deletion in the Absence of Antibiotics

### Materials and Experimental Methods

### Bacteria

L. monocytogenes strain XFL7 contains a 300 base pair deletion in the prfA gene XFL7 carries pGG55 which partially restores virulence and confers CAP resistance, and is described in United States Patent Application Publication No. 200500118184.

Development of Protocol for Plasmid Extraction from Listeria

1 mL of Listeria monocytogenes Lm-LLO-E7 research working cell bank vial was inoculated into 27 mL BH1 medium containing 34 μg/mL CAP and grown for 24 hours at 37° C. and 200 rpm.

Seven 2.5 mL samples of the culture were pelleted (15000 rpm for 5 minutes), and pellets were incubated at 37° C. with 50 μl lysozyme solution for varying amounts of time, from 0-60 minutes.

Lysozyme solution:
29 μM dibasic Potassium Phosphate
21 μM monobasic Potassium Phosphate
500 μl 40% Sucrose (filter sterilized through 0.45/μm filter)
450 μl water
60 μl lysozyme (50 mg/mL)

After incubation with the lysozyme, the suspensions were centrifuged as before and the supernatants discarded. Each pellet was then subjected to plasmid extraction by a modified version of the QlAprep Spin Miniprep Kit® (Qiagen, Germantown, Md.) protocol. The changes to the protocol were as follows:
1. The volumes of buffers PI, P2 and N3 were all increased threefold to allow complete lysis of the increased biomass.
2. 2 mg/mL of lysozyme was added to the resuspended cells before the addition of P2. The lysis solution was then incubated at 37° C. for 15 minutes before neutralization.
3. The plasmid DNA was resuspended in 30 μL rather than 50 μL to increase the concentration.

In other experiments, the cells were incubated for 15 min in P1 buffer+Lysozyme, then incubated with P2 (lysis buffer) and P3 (neutraliztion buffer) at room temperature.

Equal volumes of the isolated plasmid DNA from each subculture were run on an 0.8% agarose gel stained with ethidium bromide and visualized for any signs of structural or segregation instability.

The results showed that plasmid extraction from L. monocytogenes Lm-LLO-E7 increases in efficiency with increasing incubation time with lysozyme, up to an optimum level at approximately 50 minutes incubation.

These results provide an effective method for plasmid extraction from Listeria vaccine strains.

### Replica Plating

Dilutions of the original culture were plated onto plates containing LB or TB agar in the absence or presence of 34 μg/mL CAP. The differences between the counts on selective and non-selective agar were used to determine whether there was any gross segregational instability of the plasmid.

### Results

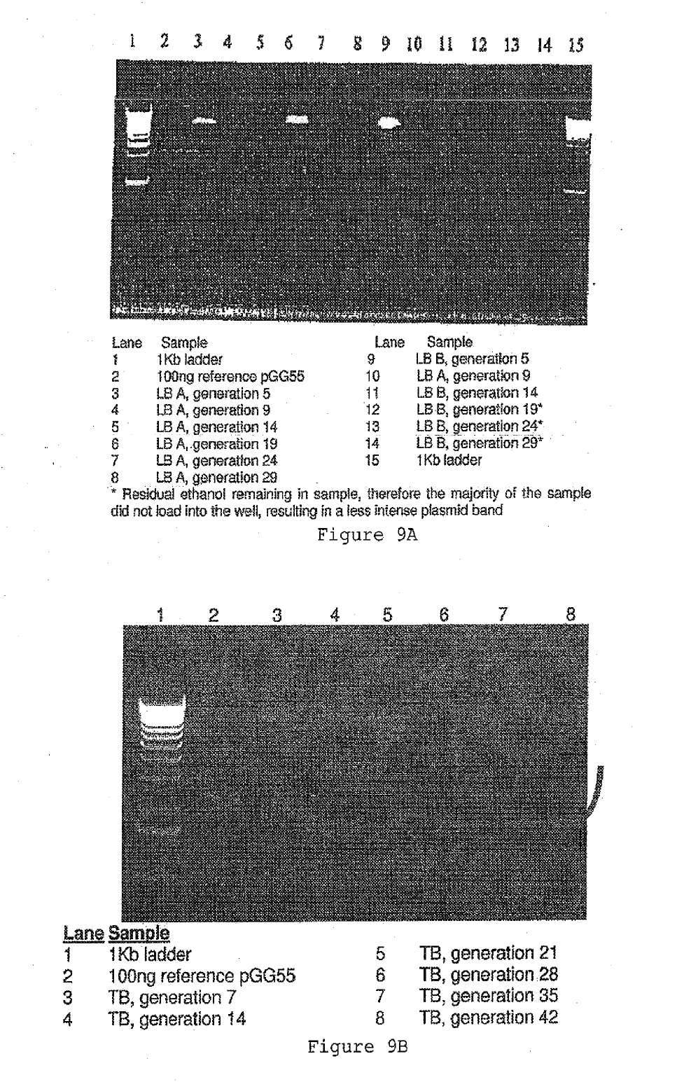

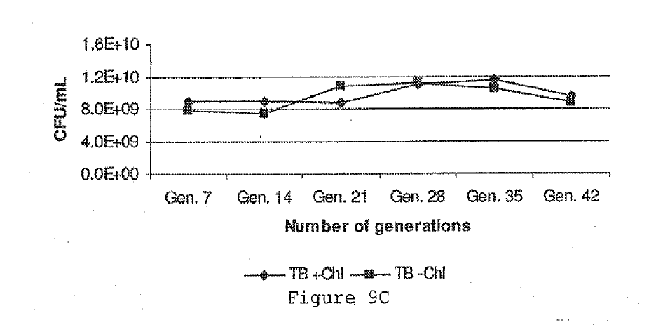

The genetic stability (i.e. the extent to which the plasmid is retained by or remains stably associated with the bacteria in the absence of selection pressure; e.g. antibiotic selection pressure) of the pGG55 plasmid in L. monocytogenes strain XFL7 in the absence of antibiotic was assessed by serial sub-culture in both Luria-Bertani media (LB: 5 g/L NaCl, 10 g/ml soy peptone, 5 g/L yeast extract) and Terrific Broth media (TB: 10 g/L glucose, 11.8 g/L soy peptone, 23.6 g/L yeast extract, 2.2 g/L KH₂PO₄, 9.4 g/L K₂HPO₄), in duplicate cultures. 50 mL of fresh media in a 250 mL baffled shake flask was inoculated with a fixed number of cells (1 ODmL), which was then subcultured at 24 hour intervals. Cultures were incubated in an orbital shaker at 37° C. and 200 rpm. At each subculture the OD₆₀₀ was measured and used to calculate the cell doubling time (or generation) elapsed, until 30 generations were reached in LB and 42 in TB. A known number of cells (15 ODmL) at each subculture stage (approximately every 4 generations) were pelleted by centrifugation, and the plasmid DNA was extracted using the Qiagen QIAprep Spin Miniprep® protocol described above. After purification, plasmid DNA was subjected to agarose gel electrophoresis, followed by ethidium bromide staining. While the amount of plasmid in the preps varied slightly between samples, the overall trend was a constant amount of plasmid with respect to the generational number of the bacteria (FIGS. 9A-B). Thus, pGG55 exhibited stability in strain XFL7, even in the absence of antibiotic.

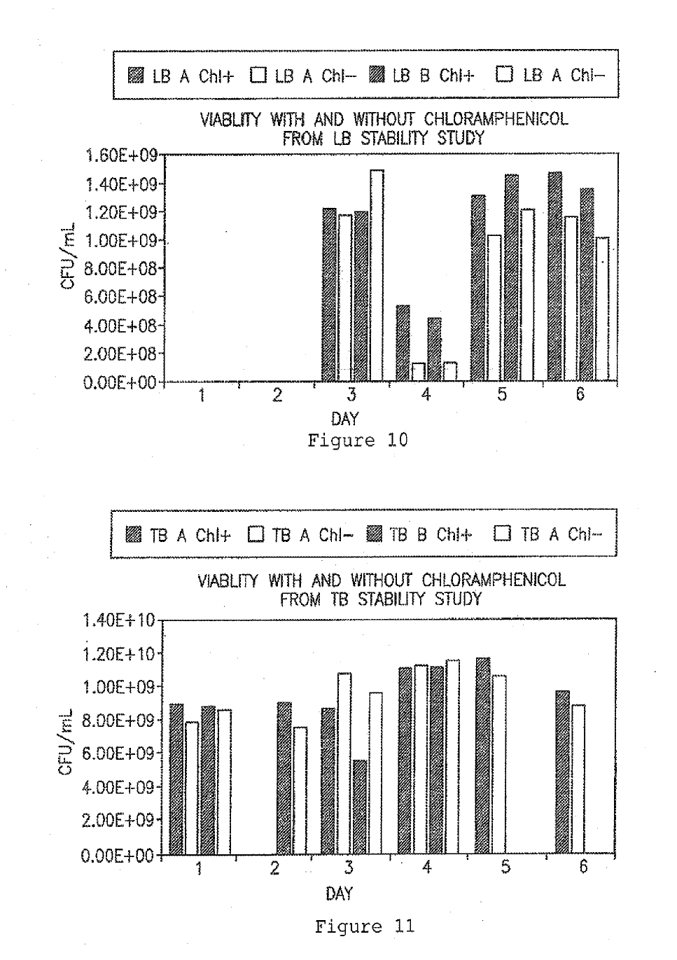

Plasmid stability was also monitored during the stability study by replica plating on agar plates at each stage of the subculture. Consistent with the results from the agarose gel electrophoresis, there was no overall change in the number of plasmid-containing cells throughout the study in either LB or TB liquid culture (FIGS. 10 and 11, respectively).

These findings demonstrate that PrfA-encoding plasmids exhibit stability in the absence of antibiotic in Listeria strains containing mutations in prfA.

### Materials and Methods (Examples 6-10)

PCR reagents:

The primers used for amplification of the prfA gene and discrimination of the D133V mutation are shown in Table 1. Stock solutions of the primers ADV451, 452 and 453 were prepared by diluting the primers in TE buffer to 400 μM. An aliquot of the stock solution was further diluted to 20 μM in water (PCR grade) to prepare a working solution. Primers were stored at −20° C. The reagents used in the PCR are shown in Table 2.

TABLE 1
Primers ADV451, 452 and 453.

| Primer | Orientation | Sequence (5′ → 3′) | Specificity |
|---|---|---|---|
| ADV451 | Forward | CCTAGCTAAATTTAATGT | D133V |
| | | (SEQ ID NO: 28) | mutation |
| ADV452 | Forward | CCTAGCTAAATTTAATGA | Wild-type |
| | | (SEQ ID NO: 29) | sequence |
| ADV453 | Reverse | TAATTTTCCCCAAGTAGCAGG | Shared |
| | | (SEQ ID NO: 30) | sequence |

TABLE 2
PCR reagents.

| | | | Catalog |
|---|---|---|---|
| | Description | Provider | number |
| 1 | 0.2 ml thin-walled PCR tubes: | Applied | N801-0612 |
| | GeneAmp autoclaved reaction | Biosystems | |
| | tube with cap | | |
| 2 | Water (PCR reagent) | Sigma | W1754 |
| 3 | Taq DNA Polymerase with 10x reaction | Sigma | D1806 |
| | buffer containing 15 mM MgCl₂ | | |
| 4 | Set of deoxynucleotides (dNTPs), | Sigma | D7295 |
| | 10 mM each | | |
| 5 | Primers ADV451, ADV452 | Invitrogen | |
| | and ADV453 | | |
| 6 | Template DNA, midipreparations | | |
| | of pGG55 plasmids | | |
| 7 | Thermal cycler PTC200 | MJ Research | |
| | (48 wells block) | | |

### Plasmid DNA Preparation

pGG55 plasmids with (pGG55 D133V) and without (pGG55 WT) the prfA mutation were extracted and purified by midipreparations either from E. coli or Listeria monocytogenes using the PureLink™ HiPure Plasmid Midiprep Kit (Invitrogen, K2100-05), according to the manufacturer's instructions. For plasmid purification from Listeria, bacterial strains carrying the pGG55 D133V or WT plasmids were streak plated from frozen stocks in BHI agar plates supplemented with chloramphenicol (25 μg/ml). A single colony from each strain was grown in 5 ml of selective medium (BHI broth with 25 μg/ml of chloramphenicol) for 6 hours with vigorous shaking at 37° C. and subinoculated 1:500 in 100 ml of selective medium for overnight growth under similar conditions. Bacteria from the overnight culture were harvested by centrifugation at 4,000×g for 10 minutes and resuspended buffer R3 (resuspension buffer) containing 2 mg/ml of lysozyme (Sigma, L7001). The bacteria suspension was incubated for at least 1 hour at 37° C. before proceeding to the regular protocol. Concentration and purity of the eluted plasmids were measured in a spectrophotometer at 260 nm and 280 nm. To prepare the template DNAs, the pGG55 D133V and WT plasmids were resuspended in water to a final concentration of 1 ng/μl from the midiprep stock solution. For the pGG55 WT plasmid, serial 10-fold dilutions from the 1 ng/μl solution were prepared, corresponding to dilutions from 10⁴ to 10⁻⁷.

prfA Specific PCR Protocol to Test Clinical Grade Material

The reaction mixture contained 1×PCR buffer, 1.5 mM MgCl₂, 0.8 mM dNTPs, 0.4 μM of each primer, 0.05 U/μl of Taq DNA polymerase and 0.04 ng/μl of the pGG55 D133V template plasmid. For each test, 10 tubes were required and the key components in each tube in a 25 μl reaction are shown in the Table 3. For the PCR reaction, a master mix was prepared with enough reagents for 11 reactions as shown in Table 4, and 24 μl of this PCR mix was added to each tube. Subsequently, a total of 1 μl of the serially diluted pGG55 WT plasmid was added to the corresponding tubes: 1 ng in tube 3; 100 pg in tube 4; 10 pg in tube 5; 1 pg in tube 6; 100 fg in tube 7; 10 fg in tube 8; 1 fg in tube 9; 0.1 fg in tube 10. This serial dilution was used to calibrate a standard curve to determine the method sensitivity. Additionally, 0.5 μl of water and 0.5 μl of primer ADV451 (20 μM stock) were added in tube 1, and 1 μl of water added in tube 2, completing 25 μl of final volume. The quantities of each reagent per tube for a 25 μl reaction are shown in Table 5. The PCR cycling conditions used in the reaction are shown in Table 6.

After conclusion of the PCR reaction, 5 μl of gel-loading buffer (6×, with bromophenol blue) was added to each sample and 10 μl were analyzed by electrophoresis in 1.2% agarose gel in TBE buffer. The gel dimensions were 7 cm×7 cm×1 cm with a 15 sample wells (1 mm×2 mm) comb. The gel was run at 100 V for ˜30 minutes, until the bromophenol blue dye reached the middle of the gel. The gel was stained in ethidium bromide (0.5 μg/ml) for 20 minutes, destaining in water for 10 minutes. The gel is visualized by illumination with UV light and photographed. The image was analyzed using a band densitometry software (Quantity One version 4.5.1, BioRad).

TABLE 3
Set of individual PCR reactions to validate the method to detect
the presence of wild-type prfA sequence in Lm-LLO-E7 samples.

| | | | | | Expected |
|---|---|---|---|---|---|
| Tube | Primer A | Primer B | Template DNA | Function | result |
| 1 | ADV451 | ADV453 | 1 ng of pGG55 | Positive control for | Positive |
| | | | (D133V) | the ADV451 reaction | |
| 2 | ADV452 | ADV453 | 1 ng of pGG55 | Negative control for | Negative |
| | | | (D133V) | the ADV452 reaction | |
| | | | | (specificity) | |
| 3 | ADV452 | ADV453 | 1 ng of pGG55 | Positive control for | Positive |
| | | | (wild-type) + 1 ng | the ADV452 reaction | |
| | | | of pGG55 (D133V) | | |
| 4 | ADV452 | ADV453 | 100 pg of pGG55 | Test the sensitivity of | Positive |
| | | | (wild-type) + 1 ng | the reaction | |
| | | | of pGG55 (D133V) | | |
| 5 | ADV452 | ADV453 | 10 pg of pGG55 | Test the sensitivity of | Positive |
| | | | (wild-type) + 1 ng | the reaction | |
| | | | of pGG55 (D133V) | | |
| 6 | ADV452 | ADV453 | 1 pg of pGG55 | Test the sensitivity of | Positive |
| | | | (wild-type) + 1 ng | the reaction | |
| | | | of pGG55 (D133V) | | |
| 7 | ADV452 | ADV453 | 100 fg of pGG55 | Test the sensitivity of | Positive |
| | | | (wild-type) + 1 ng | the reaction | |
| | | | pGG55 (D133V) | | |
| 8 | ADV452 | ADV453 | 10 fg of pGG55 | Test the sensitivity of | Positive |
| | | | (wild-type) + | the reaction | |
| | | | pGG55 (D133V) | | |
| 9 | ADV452 | ADV453 | 1 fg of pGG55 | Test the sensitivity of | Weakly |
| | | | (wild-type) + | the reaction | positive |
| | | | pGG55 (D133V) | | |
| 10 | ADV452 | ADV453 | 0.1 fg of pGG55 | Test the sensitivity of | To be |
| | | | (wild-type) + | the reaction | determined |
| | | | pGG55 (D133V) | | |

TABLE 4
Master PCR mix preparation.

| Reagent | Quantity (μl) |
|---|---|
| Water | 206.25 |
| Taq DNA Polymerase 10x reaction buffer | 27.5 |
| containing 15 mM MgCl₂ | |
| Deoxynucleotides (dNTPs) 10 mM each | 5.5 |
| Primers ADV452 (20 μM in water) | 5.5 |
| Primers ADV453 (20 μM in water) | 5.5 |
| pGG55 D133V (Lm-LLO-E7) plasmid (1 ng/μl) | 11 |
| Taq DNA Polymerase (5 U/μl) | 2.75 |
| Total | 264 |

TABLE 5
PCR protocol for validation of the method to detect the presence
of wild-type prfA sequence using primers ADV451, 452 and 453.

| Reagent | PCR |
|---|---|
| Water | 18.75 μl |
| PCR Buffer 10x + MgCl₂ 15 mM | 2.5 μl |
| Deoxynucleotides mix (dATP, dCTP, dGTP and dTTP) | 0.5 μl |
| 10 mM each | |
| Primer ADV452 (20 μM) | 0.5 μl |
| Primer ADV453 (20 μM) | 0.5 μl |
| Taq DNA polymerase (5 U/μl) | 0.25 μl |
| Template DNA (1 ng/μl) pGG55 D133V | 1 μl |
| Template DNA pGG55 WT (tubes 3 to 10)^{a} | 1 μl |
| Final volume per tube^{b} | 25 μl |

^{a}pGG55 WT (1 ng in tube 3; 100 pg in tube 4; 10 pg in tube 5; 1 pg in tube 6; 100 fg in tube 7; 10 fg in tube 8; 1 fg in tube 9; 0.1 fg in tube 10).
^{b}In tube 1, add 0.5 μl of water and 0.5 μl of primer ADV451 (20 μM stock); in tube 2 add 1 μl of water.

TABLE 6
PCR cycling conditions to detect the presence of wild-
type prfA sequence using primers ADV451, 452 and 453.

| Step | Temperature | Time | | Number of cycles |
|---|---|---|---|---|
| 1. | 94° C. | 2 minutes and 30 seconds | | 1 |
| 2. | 94° C. | 30 | seconds | 1 |
| 3. | 53° C. | 30 | seconds | 1 |
| 4. | 72° C. | 30 | seconds | 1 |
| 5. | Repeat steps 2 to 4 | | | 12 |
| 6. | 94° C. | 30 | seconds | 1 |
| 7. | 50° C. | 30 | seconds | 1 |
| 8. | 72° C. | 30 | seconds | 1 |
| 9. | Repeat steps 6 to 8 | | | 23 |
| 10. | 72° C. | 10 | minutes | 1 |

### Sequencing:

Sequencing of the plasmids was done using the dideoxy sequencing method. The plasmids pGG55 D133V and pGG55 WT were mixed at different ratios (1:1, 1:10, 1;100, 1:1,000 and 1:10,000). The total amount of plasmid in the mixture was kept constant (500 μg) and the plasmid containing the wild-type sequence was 10-fold serially diluted in relation to the D133V plasmid to determine the sensitivity of the method.

### Results

### EXAMPLE 6

### Sequencing is Not a Sensitive Method to Detect the Reversion of the D133V Mutation

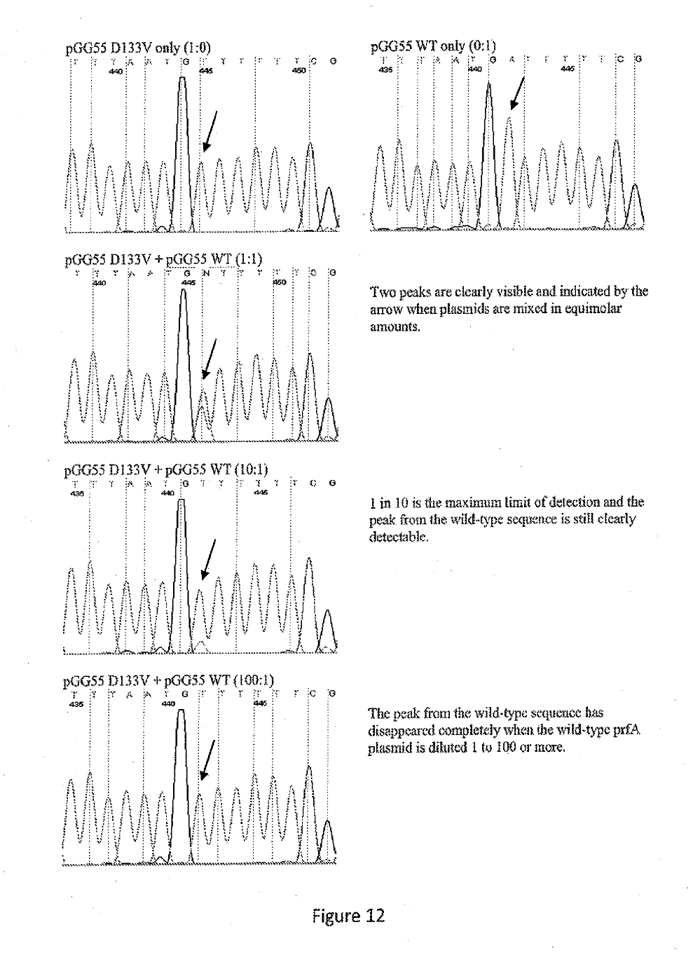

To estimate the sensitivity of sequencing in detecting the wild-type prfA sequence, the pGG55 D133V and WT plasmids were mixed at the different ratios and sequenced. The results are shown in FIG. 12 and reveal that sequencing has a high specificity in discriminating the prfA D133V mutation (FIG. 12). On the other hand, the sensitivity is low and the maximum dilution of wild-type prfA pGG55 plasmid with a detectable peak in the sequence was 1 in 10 (FIG. 12). In conclusion, although sequencing is very specific, the sensitivity of the method is low and not appropriate to screen for the presence of rare events such as revertants of the prfA D133V mutation in Lm-LLO-E7 samples.

### EXAMPLE 7

### Development of a Highly Specific and Sensitive PCR Method to Detect Reversion of the D133V Mutation

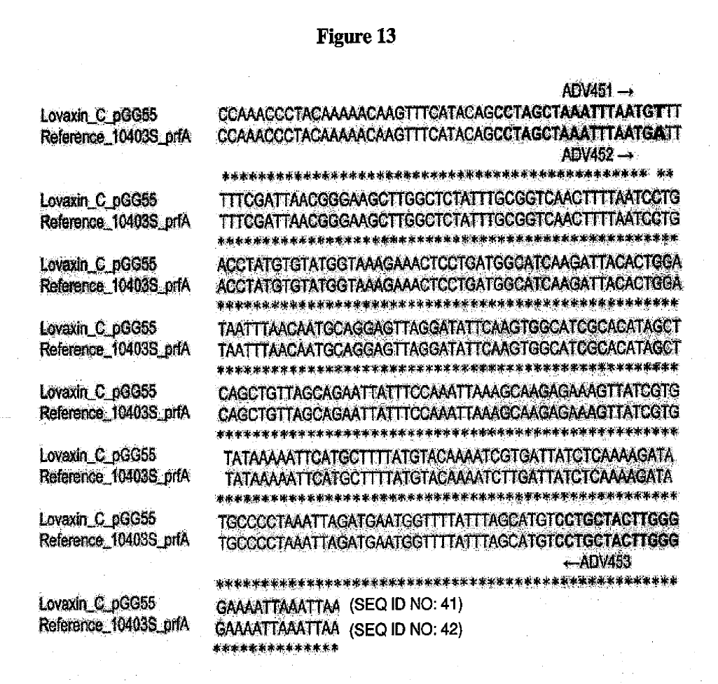

Given the low sensitivity of sequencing to detect rare events, it became imperative to develop a more sensitive method with similar specificity to detect reversion of the D133V mutation to wild-type. To achieve this goal, we designed a PCR-based method that specifically amplifies the wild-type sequence and is sensitive enough to detect at least 1 wild-type copy of prfA in 10,000,000 copies of the D133V mutated sequence. We designed 3 primers for this method: ADV451, ADV452 and ADV453 (Table 1). Both ADV451 and ADV452 are forward primers and differ in the last nucleotide at the 3′ position to discriminate the A→T (D133V) mutation at position 398 of the prfA gene. The ADV453 primer is the reverse primer located approximately 300 bp downstream the annealing site of the ADV451 and ADV452 primers (FIG. 13). The expected PCR band obtained with the primers ADV451 or ADV452 and ADV453 is 326 bp. Under stringent conditions, the ADV451 primer should only amplify the pGG55 D133V plasmid, whereas the ADV452 would be specific to the wild-type prfA sequence.

### EXAMPLE 8

### Specificity of the PCR Method

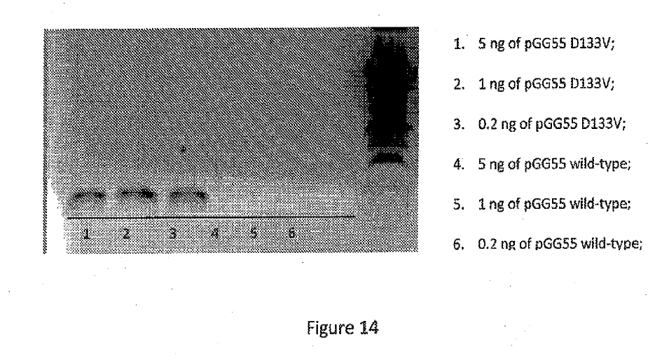

The reaction using the primer ADV451 was very specific and amplified the mutated D133V prfA sequence (lanes 1 to 3), but not the wild-type sequence (lanes 4 to 6). However, a very faint band can be detected in lane 4, when 5 ng of template DNA was used, but not with 1 ng (FIG. 14).

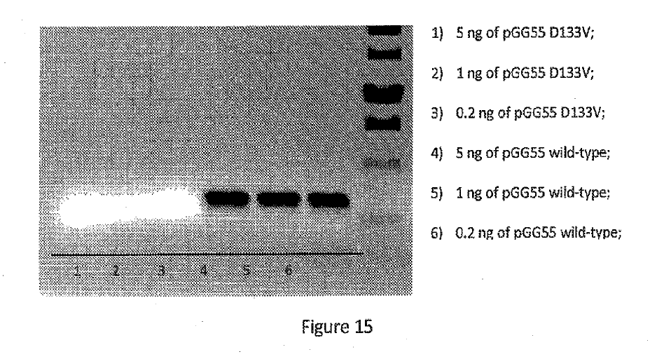

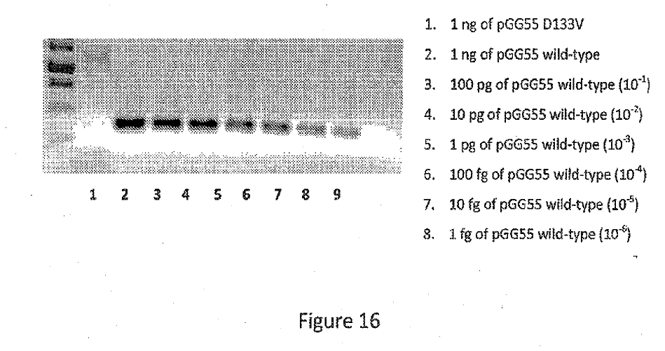

As shown in FIG. 15, the reaction with the ADV452 primer only amplified the wild-type prfA sequence (lanes 4, 5 and 6), and no bands were detected when the pGG55 carrying the D133V PrfA mutation was used as a template (lanes 1, 2 and 3), even when using 5 ng of plasmid in the reaction (FIG. 16). In conclusion, the PCR reactions with primers ADV451 and ADV452 are very specific and able to discriminate the A↔T (D133V) mutation at position 398 of the prfA gene in the pGG55 plasmid. Based on these results, we selected the amount of 1 ng as the standard amount of template DNA to be used in the reaction.

### EXAMPLE 9

### Sensitivity of the PCR Method

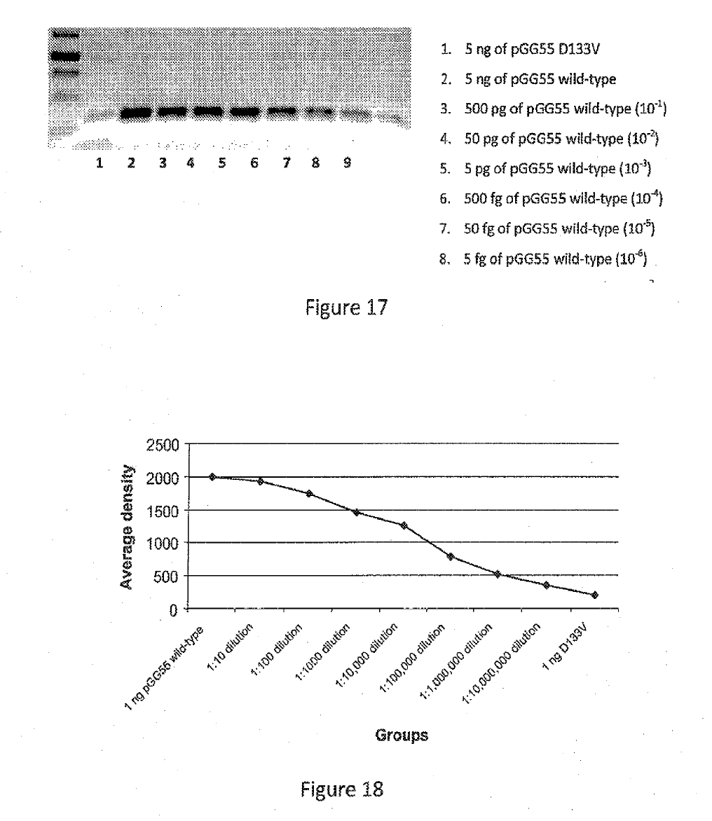

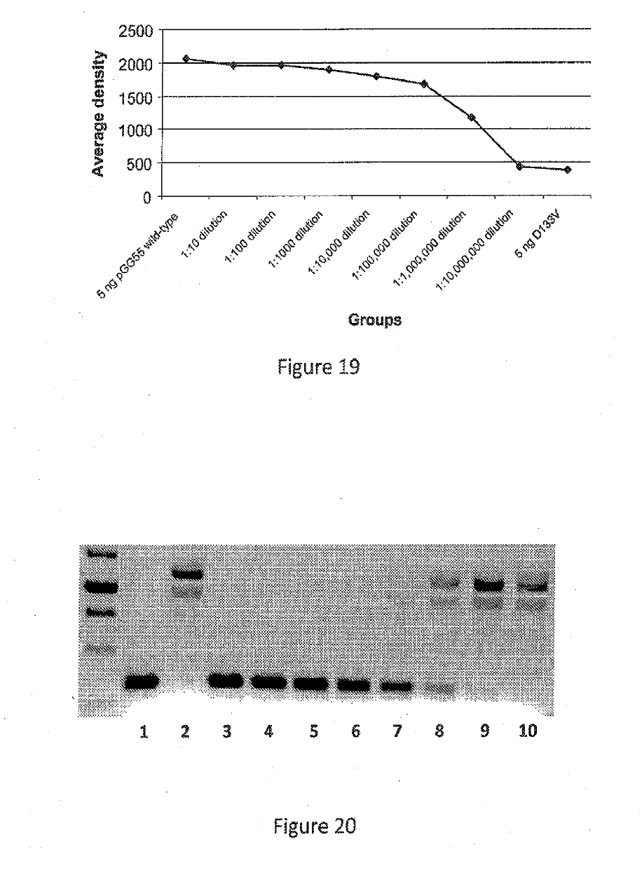

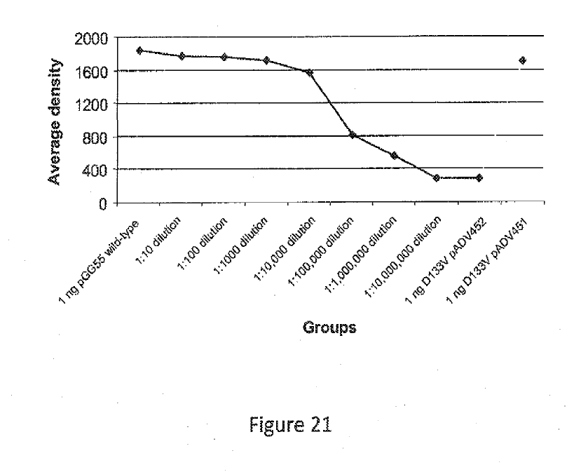

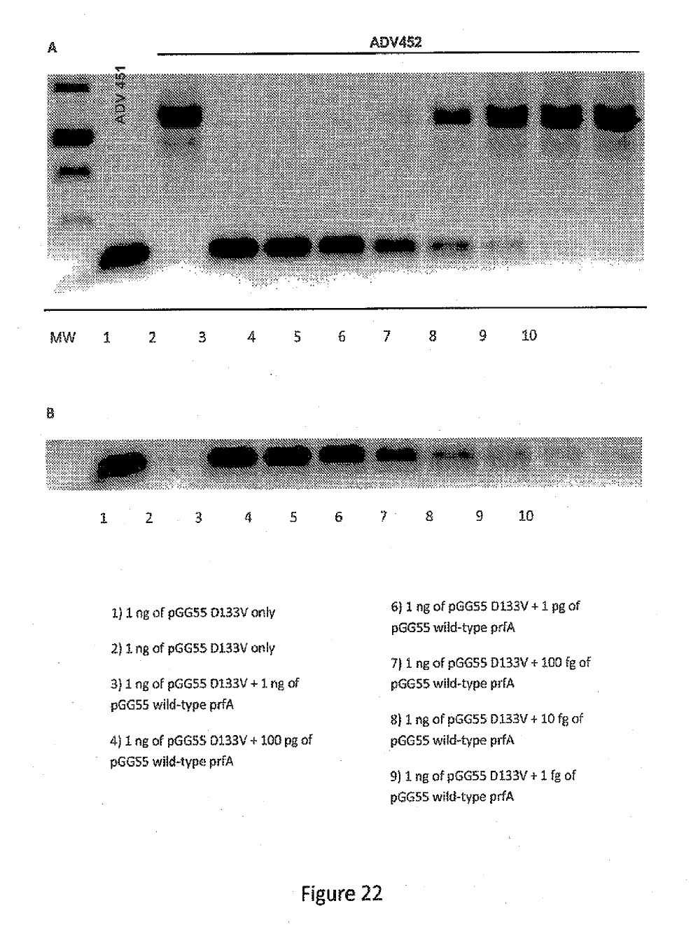

The sensitivity of the reaction was tested using 1 ng of template DNA. For the plasmid carrying the wild-type prfA sequence, decreasing amounts of DNA (corresponding to 10-fold dilutions from 10⁻¹ to 10⁻⁷), were also included in the reaction to estimate the sensitivity. In these reactions only the primers ADV452 and ADV453 were used. In a PCR reaction with 30 cycles (10 cycles with annealing temperature of 53° C. and an additional 20 cycles with annealing temperature of 50° C.), the sensitivity of the method was 1 in 100,000 (data not shown). As shown in FIG. 5, increasing the number of PCR cycles to 37 improved the visual sensitivity of the method to 10⁻⁶ for the detection of D133V revertants, without significantly compromising the specificity. A clear band was visible at the 10⁻⁶ dilution, corresponding to a detection level of 1 copy of the wild-type sequence in a million of the D133V mutant, when 1 ng of plasmid was used as the initial amount of DNA. Only a very weak band can be visualized in lanes 1 and 9 after longer exposure, reassuring the robust specificity of the method. On the other hand, when starting with 5 ng of DNA, a band could be easily detected at the 10⁻⁷ dilution, increasing the sensitivity of the PCR. However, a similar band in intensity could also be detected with the pGG55 D133V plasmid, indicating the specificity limit of the method (FIG. 17). This band observed with the pGG55 D133V plasmid is likely due to non-specific amplification of the D133V mutation with primer ADV452 that can significantly accumulate with the increased number of cycles. These results indicate that the sensitivity limit for this method, without significantly compromising the specificity, is situated between 1 to 1,000,000 and 1 to 10,000,000.

### EXAMPLE 10

### Recombinant Listeria Expressing a Fusion Protein of LLO to E7(Lm-LLO-E7)

This strain is approx. 4-5 logs more attenuated than the wild-type parent strain 10403S and secretes the fusion protein tLLO-E7. This immunotherapy is based on the backbone XFL7, which is derived from 10403S by the irreversible deletion in the virulence gene transcription activator prfA. PrfA regulates the transcription of several virulence genes such as Listeriolysin O (LLO), ActA, PlcA (phospholipase A), PlcB (phospholipase B) etc that are required for in vivo intracellular growth and survival of L. monocytogenes. The plasmid pGG55 is retained by the Lm-LLO-E7 in vitro by means of selection with ‘chloramphenicol’. However for in vivo retention of the plasmid by Lm-LLO-E7, it carries a copy of mutated prfA (D133V), which has been demonstrated to be less active than wild-type PrfA in DNA binding and activating the transcription of virulence genes. We have observed that complementation with mutated PrfA resulted in approx. 40 fold reduction in the amount of secreted LLO from Lm-LLO-E7 when compared to wild-type strain 10403S. This implicates that possibly the strain Lm-LLO-E7 exhibits a reduced expression of the virulence genes that are regulated by PrfA such as actA, inlA, inlB, inlC, plcB etc. In Lm-LLO-E7, the complementation with mutated copy of prfA possibly causes a reduction in the expression of different virulence genes that are regulated by PrfA resulting in overall attenuation of approx. 4-5 logs.

### EXAMPLE 11

### Construction of Attenuated Listeria Strain-LmddΔactA

A recombinant Lm that secretes PSA fused to tLLO (Lm-LLO-PSA) was developed, which elicits a potent PSA-specific immune response associated with regression of tumors in a mouse model for prostate cancer, wherein the expression of tLLO-PSA is derived from a plasmid based on pGG55 (Table 7), which confers antibiotic resistance to the vector. for a strain for the PSA vaccine based on the pADV142 plasmid was also developed. This strain, has no antibiotic resistance markers, and is referred as LmddA-142 (Table 7). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 7
Plasmids and strains

| Plasmids | Features |
|---|---|
| pGG55 | pAM401/pGB354 shuttle plasmid with gram(−) and gram(+) |
| | cm resistance, LLO-E7 expression cassette and a copy of |
| | LmprfA gene |
| pTV3 | Derived from pGG55 by deleting cm genes and inserting the |
| | Lmdal gene |
| pADV119 | Derived from pTV3 by deleting the prfA gene |
| pADV134 | Derived from pADV119 by replacing the Lmdal gene by the |
| | Bacillusdal gene |
| pADV142 | Derived from pADV134 by replacing HPV16 e7 with klk3 |
| pADV172 | Derived from pADV134 by replacing HPV16 e7 with hmw- |
| | maa₂₁₆₀₋₂₂₅₈ |

| Strains | Genotype |
|---|---|
| 10403S | Wild-typeListeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly gene in |
| | the chromosome |
| LmddA-143 | 10403S dal⁽⁻⁾ dat⁽⁻⁾ actA⁽⁻⁾ with klk3 fused to the hly |
| | gene in the chromosome |
| LmddA-172 | 10403S dal⁽⁻⁾ dat⁽⁻⁾ actA⁽⁻⁾ pADV172 |
| Lmdd-143/134 | Lmdd-143 pADV134 |
| LmddA-143/134 | LmddA-143 pADV134 |
| Lmdd-143/172 | Lmdd-143 pADV172 |
| LmddA-143/172 | LmddA-143 pADV172 |

The sequence of the plasmid pAdv142 (6523 bp) was as follows:

(SEQ ID NO: 34)

cggagtgtatactggcttactatgttggcactgatgagggtgtcagtgaa
gtgcttcatgtggcaggagaaaaaaggctgcaccggtgcgtcagcagaat
atgtgatacaggatatattccgcttcctcgctcactgactcgctacgctc
ggtcgttcgactgcggcgagcggaaatggcttacgaacggggcggagatt
tcctggaagatgccaggaagatacttaacagggaagtgagagggccgcgg
caaagccgtttttccataggctccgcccccctgacaagcatcacgaaatc
tgacgctcaaatcagtggtggcgaaacccgacaggactataaagatacca
ggcgtttccccctggcggctccctcgtgcgctctcctgttcctgcctttc
ggtttaccggtgtcattccgctgttatggccgcgtttgtctcattccacg
cctgacactcagttccgggtaggcagttcgctccaagctggactgtatgc
acgaaccccccgttcagtccgaccgctgcgccttatccggtaactatcgt
cttgagtccaacccggaaagacatgcaaaagcaccactggcagcagccac
tggtaattgatttagaggagttagtcttgaagtcatgcgccggttaaggc
taaactgaaaggacaagttttggtgactgcgctcctccaagccagttacc
tcggttcaaagagttggtagctcagagaaccttcgaaaaaccgccctgca
aggcggttttttcgttttcagagcaagagattacgcgcagaccaaaacga
tctcaagaagatcatcttattaatcagataaaatatttctagccctcctt
tgattagtatattcctatcttaaagttacttttatgtggaggcattaaca
tttgttaatgacgtcaaaaggatagcaagactagaataaagctataaagc
aagcatataatattgcgtttcatctttagaagcgaatttcgccaatatta
taattatcaaaagagaggggtggcaaacggtatttggcattattaggtta
aaaaatgtagaaggagagtgaaacccatgaaaaaaataatgctagttttt
attacacttatattagttagtctaccaattgcgcaacaaactgaagcaaa
ggatgcatctgcattcaataaagaaaattcaatttcatccatggcaccac
cagcatctccgcctgcaagtcctaagacgccaatcgaaaagaaacacgcg
gatgaaatcgataagtatatacaaggattggattacaataaaaacaatgt
attagtataccacggagatgcagtgacaaatgtgccgccaagaaaaggtt
acaaagatggaaatgaatatattgttgtggagaaaaagaagaaatccatc
aatcaaaataatgcagacattcaagttgtgaatgcaatttcgagcctaac
ctatccaggtgctctcgtaaaagcgaattcggaattagtagaaaatcaac
cagatgttctccctgtaaaacgtgattcattaacactcagcattgatttg
ccaggtatgactaatcaagacaataaaatagttgtaaaaaatgccactaa
atcaaacgttaacaacgcagtaaatacattagtggaaagatggaatgaaa
aatatgctcaagcttatccaaatgtaagtgcaaaaattgattatgatgac
gaaatggcttacagtgaatcacaattaattgcgaaatttggtacagcatt
taaagctgtaaataatagcttgaatgtaaacttcggcgcaatcagtgaag
ggaaaatgcaagaagaagtcattagttttaaacaaatttactataacgtg
aatgttaatgaacctacaagaccttccagatttttcggcaaagctgttac
taaagagcagttgcaagcgcttggagtgaatgcagaaaatcctcctgcat
atatctcaagtgtggcgtatggccgtcaagtttatttgaaattatcaact
aattcccatagtactaaagtaaaagctgcttttgatgctgccgtaagcgg
aaaatctgtctcaggtgatgtagaactaacaaatatcatcaaaaattctt
ccttcaaagccgtaatttacggaggttccgcaaaagatgaagttcaaatc
atcgacggcaacctcggagacttacgcgatattttgaaaaaaggcgctac
ttttaatcgagaaacaccaggagttcccattgcttatacaacaaacttcc
taaaagacaatgaattagctgttattaaaaacaactcagaatatattgaa
acaacttcaaaagcttatacagatggaaaaattaacatcgatcactctgg
aggatacgttgctcaattcaacatttcttgggatgaagtaaattatgatc
tcgagattgtgggaggctgggagtgcgagaagcattcccaaccctggcag
gtgcttgtggcctctcgtggcagggcagtctgcggcggtgttctggtgca
cccccagtgggtcctcacagctgcccactgcatcaggaacaaaagcgtga
tcttgctgggtcggcacagcctgtttcatcctgaagacacaggccaggta
tttcaggtcagccacagcttcccacacccgctctacgatatgagcctcct
gaagaatcgattcctcaggccaggtgatgactccagccacgacctcatgc
tgctccgcctgtcagagcctgccgagctcacggatgctgtgaaggtcatg
gacctgcccacccaggagccagcactggggaccacctgctacgcctcagg
ctggggcagcattgaaccagaggagttcttgaccccaaagaaacttcagt
gtgtggacctccatgttatttccaatgacgtgtgtgcgcaagttcaccct
cagaaggtgaccaagttcatgctgtgtgctggacgctggacagggggcaa
aagcacctgctcgggtgatttctgggggcccacttgtctgttatggtgtg
cttcaaggtatcacgtcatggggcagtgaaccatgtgccctgcccgaaag
gccttccctgtacaccaaggtggtgcattaccggaagtggatcaaggaca
ccatcgtggccaaccccTAAcccgggccactaactcaacgctagtagtgg
atttaatcccaaatgagccaacagaaccagaaccagaaacagaacaagta
acattggagttagaaatggaagaagaaaaaagcaatgatttcgtgtgaat
aatgcacgaaatcattgcttatttttttaaaaagcgatatactagatata
acgaaacaacgaactgaataaagaatacaaaaaaagagccacgaccagtt
aaagcctgagaaactttaactgcgagccttaattgattaccaccaatcaa
ttaaagaagtcgagacccaaaatttggtaaagtatttaattactttatta
atcagatacttaaatatctgtaaacccattatatcgggtttttgagggga
tttcaagtctttaagaagataccaggcaatcaattaagaaaaacttagtt
gattgccttttttgttgtgattcaactttgatcgtagcttctaactaatt
aattttcgtaagaaaggagaacagctgaatgaatatcccttttgttgtag
aaactgtgcttcatgacggcttgttaaagtacaaatttaaaaatagtaaa
attcgctcaatcactaccaagccaggtaaaagtaaaggggctatttttgc
gtatcgctcaaaaaaaagcatgattggcggacgtggcgttgttctgactt
ccgaagaagcgattcacgaaaatcaagatacatttacgcattggacacca
aacgtttatcgttatggtacgtatgcagacgaaaaccgttcatacactaa
aggacattctgaaaacaatttaagacaaatcaataccttctttattgatt
ttgatattcacacggaaaaagaaactatttcagcaagcgatattttaaca
acagctattgatttaggttttatgcctacgttaattatcaaatctgataa
aggttatcaagcatattttgttttagaaacgccagtctatgtgacttcaa
aatcagaatttaaatctgtcaaagcagccaaaataatctcgcaaaatatc
cgagaatattttggaaagtctttgccagttgatctaacgtgcaatcattt
tgggattgctcgtataccaagaacggacaatgtagaattttttgatccca
attaccgttattctttcaaagaatggcaagattggtctttcaaacaaaca
gataataagggctttactcgttcaagtctaacggttttaagcggtacaga
aggcaaaaaacaagtagatgaaccctggtttaatctcttattgcacgaaa
cgaaattttcaggagaaaagggtttagtagggcgcaatagcgttatgttt
accctctctttagcctactttagttcaggctattcaatcgaaacgtgcga
atataatatgtttgagtttaataatcgattagatcaacccttagaagaaa
aagaagtaatcaaaattgttagaagtgcctattcagaaaactatcaaggg
gctaatagggaatacattaccattctttgcaaagcttgggtatcaagtga
tttaaccagtaaagatttatttgtccgtcaagggtggtttaaattcaaga
aaaaaagaagcgaacgtcaacgtgttcatttgtcagaatggaaagaagat
ttaatggcttatattagcgaaaaaagcgatgtatacaagccttatttagc
gacgaccaaaaaagagattagagaagtgctaggcattcctgaacggacat
tagataaattgctgaaggtactgaaggcgaatcaggaaattttctttaag
attaaaccaggaagaaatggtggcattcaacttgctagtgttaaatcatt
gttgctatcgatcattaaattaaaaaaagaagaacgagaaagctatataa
aggcgctgacagcttcgtttaatttagaacgtacatttattcaagaaact
ctaaacaaattggcagaacgccccaaaacggacccacaactcgatttgtt
tagctacgatacaggctgaaaataaaacccgcactatgccattacattta
tatctatgatacgtgtttgtttttctttgctggctagcttaattgcttat
atttacctgcaataaaggatttcttacttccattatactcccattttcca
aaaacatacggggaacacgggaacttattgtacaggccacctcatagtta
atggtttcgagccttcctgcaatctcatccatggaaatatattcatcccc
ctgccggcctattaatgtgacttttgtgcccggcggatattcctgatcca
gctccaccataaattggtccatgcaaattcggccggcaattttcaggcgt
tttcccttcacaaggatgtcggtccctttcaattttcggagccagccgtc
cgcatagcctacaggcaccgtcccgatccatgtgtctttttccgctgtgt
actcggctccgtagctgacgctctcgccttttctgatcagtttgacatgt
gacagtgtcgaatgcagggtaaatgccggacgcagctgaaacggtatctc
gtccgacatgtcagcagacgggcgaaggccatacatgccgatgccgaatc
tgactgcattaaaaaagccttttttcagccggagtccagcggcgctgttc
gcgcagtggaccattagattctttaacggcagcggagcaatcagctcttt
aaagcgctcaaactgcattaagaaatagcctctttctttttcatccgctg
tcgcaaaatgggtaaatacccctttgcactttaaacgagggttgcggtca
agaattgccatcacgttctgaacttcttcctctgtttttacaccaagtct
gttcatccccgtatcgaccttcagatgaaaatgaagagaaccttttttcg
tgtggcgggctgcctcctgaagccattcaacagaataacctgttaaggtc
acgtcatactcagcagcgattgccacatactccgggggaaccgcgccaag
caccaatataggcgccttcaatccctttttgcgcagtgaaatcgcttcat
ccaaaatggccacggccaagcatgaagcacctgcgtcaagagcagccttt
gctgtttctgcatcaccatgcccgtaggcgtttgctttcacaactgccat
caagtggacatgttcaccgatatgttttttcatattgctgacattttcct
ttatcgcggacaagtcaatttccgcccacgtatctctgtaaaaaggtttt
gtgctcatggaaaactcctctcttttttcagaaaatcccagtacgtaatt
aagtatttgagaattaattttatattgattaatactaagtttacccagtt
ttcacctaaaaaacaaatgatgagataatagctccaaaggctaaagagga
ctataccaactatttgttaattaa.

This plasmid was sequenced at Genewiz facility from the E. coli strain on Feb. 20, 2008.

### EXAMPLE 12

### Insertion of the Human klk3 Gene in Frame to the hly Gene In The Lmdd And Lmdda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal datΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 8. The upstream and downstream DNA regions of actA were cloned in the pNEB193 at the EcoRI/PstIrestriction site and from this plasmid, the EcoRI/PstIwas further cloned in the temperature sensitive plasmid pKSV7, resulting in ΔactA/pKSV7 (pAdv120).

TABLE 8
Sequence of primers that was used for the
amplification of DNA sequences upstream
and downstream of actA

| Primer | Sequence | SEQ ID NO: |
|---|---|---|
| Adv271-actAF1 | cgGAATTCGGATCCgcgccaaatcattggttgattg | 35 |
| Adv272-actAR1 | gcgaGTCGACgtcggggttaatcgtaatgcaattggc | 36 |
| Adv273-actAF2 | gcgaGTCGACccatacgacgttaattcttgcaatg | 37 |
| Adv274-actAR2 | gataCTGCAGGGATCCttcccttctcggtaatcagtcac | 38 |

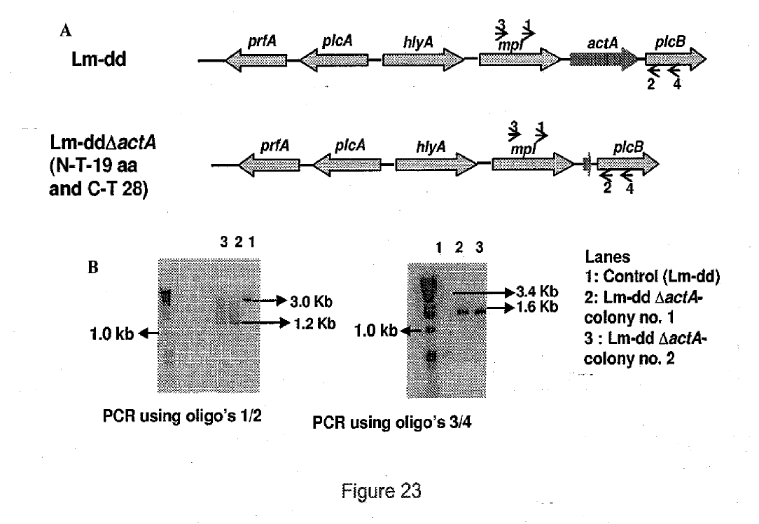

The deletion of the gene from its chromosomal location was verified using primers that bind externally to the actA deletion region, which are shown in FIG. 23 as primer 3 (Adv 305-tgggatggccaagaaattc, SEQ ID NO: 39) and primer 4 (Adv304-ctaccatgtcttccgttgcttg; SEQ ID NO: 40). The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIG. 23 confirms that the 1.8 kb region of actA was deleted in the LmddΔactA strain. DNA sequencing was also performed on PCR products to confirm the deletion of actA containing region in the strain, LmddΔactA.

### EXAMPLE 13

### Construction of the Antibiotic-Independent Episomal Expression System for Antigen Delivery by Lm Vectors

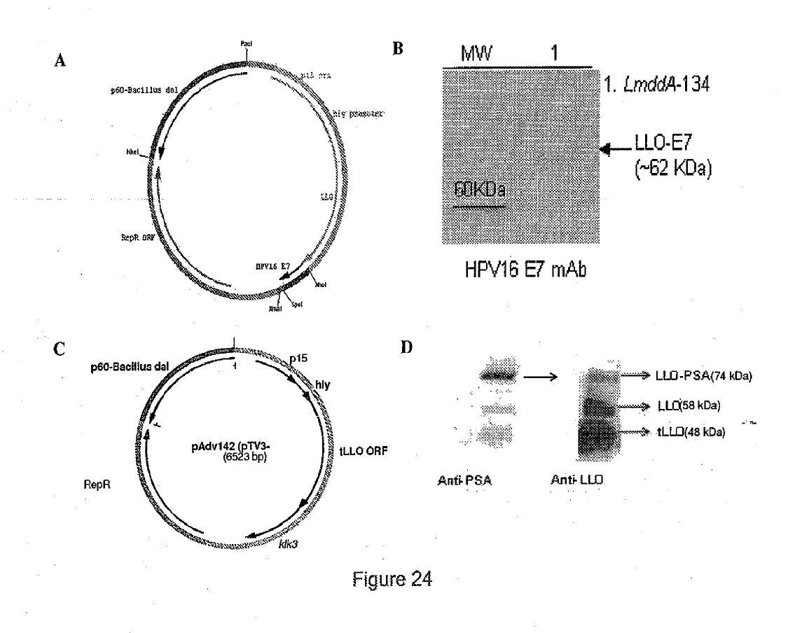

The antibiotic-independent episomal expression system for antigen delivery by Lm vectors (pAdv142) is the next generation of the antibiotic-free plasmid pTV3 (Verch et al., Infect Immun, 2004. 72 (11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 24A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 24B). The Lmdd system derived from the 10403 S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 24C, Table 7) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 24C).

The plasmid pAdv142 was transformed to the Listeria background strains, Lmdd actA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 24D). There was stable expression and secretion of LLO-PSA fusion protein by the strain, Lm-ddA-LLO-PSA after two in vivo passages.

### EXAMPLE 12

### TC1 Tumor Control After Immunization with Various Dose Concentrations for LMDDAHPVQUAD-Tag

Tumor regression study in C57BL/6 female mice using TC-1 lung epithelial cells to assess the therapeutic efficacy of different doses (CFU) of Lm-HPVQuad treatment: 1×10⁸, 1×10⁷, 2.5×10⁷, and 5×10⁷.

Tumor Inoculation: The tumor model used in this study is the TC-1 tumor model. TC-1 cells are cultured in complete medium. Two days prior to implanting tumor cells in mice, TC-1 cells will be sub-cultured in complete media. On the day of the experiment (Day 0), cells will be trypsinized and washed twice with media. Cells will be counted and resuspended at a concentration of 1×10⁵ cells/200 ul in PBS/mouse for injection. Tumor cells will be injected subcutaneously in the right flank of each mouse. Dose 1 will be administered IP/200 uL/mouse on Day 8 and weekly thereafter for a total of 3 doses. Tumors will be measured twice a week until they reach a size of 12 mm in diameter. Once tumors meet sacrifice criteria, mice will be euthanized according to Advaxis' animal euthanasia protocol and tumors will be excised and measured.

Culture protocol: Two days prior to implantation, TC1 cells were sub-cultured to 1:10 ratio. On day of implantation, TC1 cells were trypsinized, and washed with c-RPMI. Cells were counted (1×10̂6/ml, 97% live, 20 ml total), and washed again in RPMI. Cells were then resuspended in ice-cold PBS, and passed through 70um strainer, and counted again (2×10̂6/ml in 10 ml, 97% live). 4 ml of cells were mixed with 12 ml of ice cold PBS to yield 5×10̂5/ml in 16 ml total. Enough for 80 animals given 1×10̂5/200 ul dose.

Complete medium for TC-1 cells: 450 ml RPMI 1640, 50 ml 10%FBS, 5 ml NEAA (100 uM), 5 ml L-Glutamine (2 uM), 5 mL Pen/strep (100 U/mL penicillin+100 ug/mL streptomycin) G418 (400 ug/mL) (add to cells when splitting).

Vaccine preparations: 1. Thaw 1 vial from −80° C. at 37° C. water bath for each construct. 2. Spin at 14,000 rpm for 2 min and discard supernatant. 3. Wash 2 times with 1 ml of PBS and discard. 4. Resuspend in PBS to appropriate concentration.

TABLE 9
Immunization schedule

| | Titer- | Mice/ | Dose- | | | |
|---|---|---|---|---|---|---|
| Vaccine/Group | CFU/mL | Group | Concentration | Dose 1 | Dose 2 | Dose 3 |
| 1) LmddA274 | 1.7 × 10⁹ | 8 | 1 × 10⁸ | Day 1 | 7 days | 7 days |
| | | | | | after | after |
| | | | | | Dose 1 | Dose 2 |
| 2) Lmdda-HPVquad-tag | 1.4 × 10⁹ | 8 | 1 × 10⁸ | Day 1 | 7 days | 7 days |
| | | | | | after | after |
| | | | | | Dose 1 | Dose 2 |
| 3) Lmdda-HPVquad-tag | 1.4 × 10⁹ | 8 | 1 × 10⁷ | Day 1 | 7 days | 7 days |
| | | | | | after | after |
| | | | | | Dose 1 | Dose 2 |
| 4) Lmdda-HPVquad-tag | 1.4 × 10⁹ | 8 | 2.5 × 10⁷ | Day 1 | 7 days | 7 days |
| | | | | | after | after |
| | | | | | Dose 1 | Dose 2 |
| 5) Lmdda-HPVquad-tag | 1.4 × 10⁹ | 8 | 5 × 10⁷ | Day 1 | 7 days | 7 days |
| | | | | | after | after |
| | | | | | Dose 1 | Dose 2 |

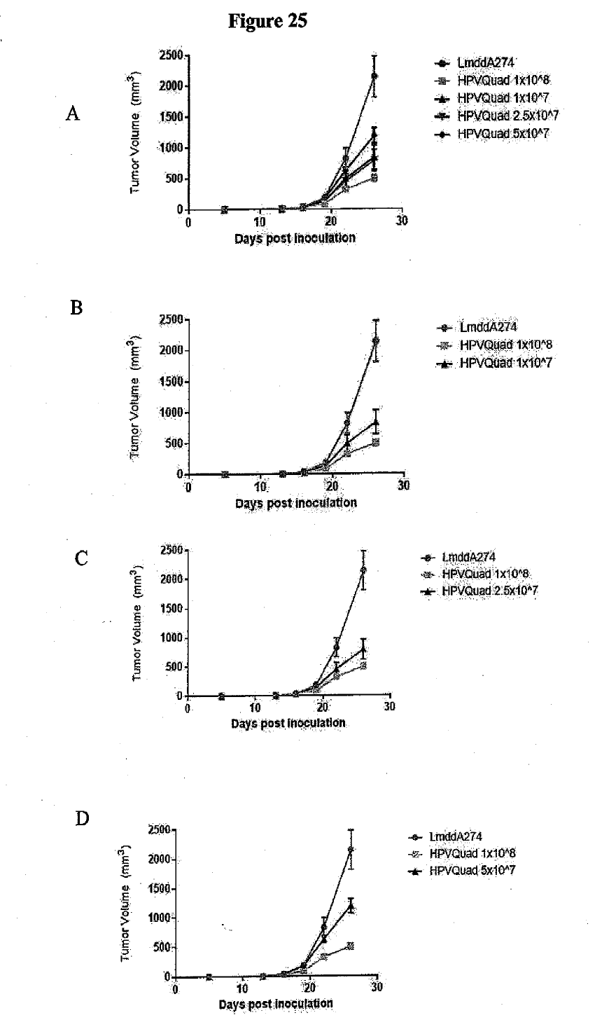

Tumor volume was measured in mice inoculated with TC1 tumors that express HPV 16 E6&E7; that were treated with decreasing doses Lmdda-HPVQuad or empty LmddA274 (Listeria without HPV-antigens) (FIG. 25A-D).

Results showed that HPVquad can be given at doses up to 10-fold less (1×10⁷) than the optimum dose of 1×10⁸ in mice and still maintain equally as effective tumor control of HPV16 E6 & E7 expressing tumors.

### EXAMPLE 13

### TC1 Tumor Control After Immunization with Various Dose Concentrations for Axal or LMDDA-HPVQUAD

Tumor regression study in C57BL/6 female mice using TC-1 lung epithelial cells (expresses HPV 16: E6&E7) to assess the therapeutic efficacy of different doses (CFU) of AXAL (Lm-E7) or Lm-HPVQuad treatment: 1×10⁸, 1×10⁷, 2.5×10⁷, and 5×10⁷. N=5-10 animals per group.

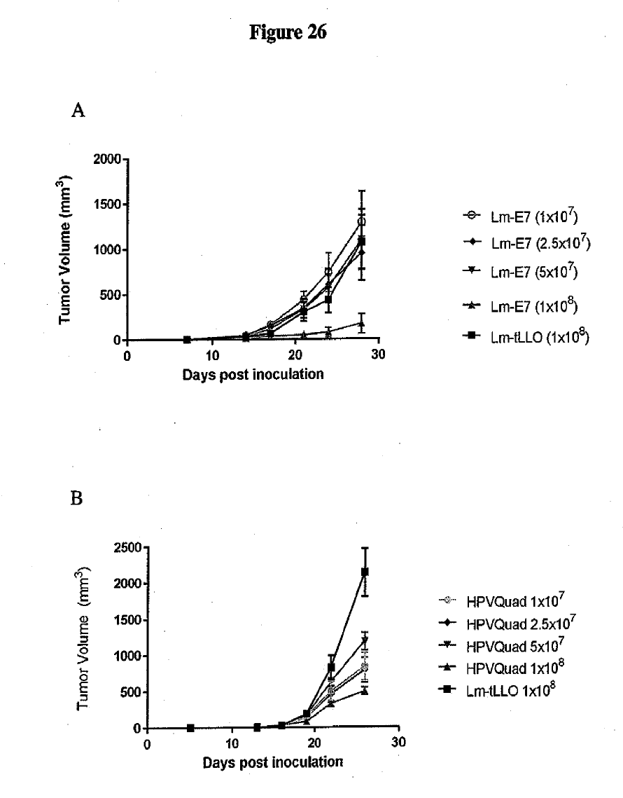

Tumor volume was measured in mice inoculated with TC1 tumors that express HPV 16 E6&E7; that were treated with decreasing doses AXAL/Lm-E7 or with an empty vector Lm-tLLO (FIG. 26A). Tumor volume was measured in mice inoculated with TC1 tumors that express HPV 16 E6&E7; that were treated with decreasing doses Lmdda-HPVQuad or with an empty vector Lm-tLLO (FIG. 26B).

Results showed that AXAL, which expresses only the HPV 16 E7 antigen, is effective at the standard dose of 1×10⁸. However, HPVQuad, which expresses both HPV16 E6 & E7, is effective at controlling tumor growth at 10-fold less than the standard dose.

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed:

1. An immunogenic composition comprising a recombinant Listeria strain comprising a recombinant nucleic acid, said nucleic acid comprising a first open reading frame encoding a recombinant polypeptide comprising four heterologous HPV antigens or functional fragments thereof operably linked in tandem, wherein said operably linked heterologous HPV antigens or functional fragments thereof are fused to an N-terminal fragment of an LLO protein at the N-terminus of the most N-terminal heterologous antigen, wherein said recombinant nucleic acid further comprises a second open reading frame encoding a metabolic enzyme.

2. The immunogenic composition of claim 1, wherein said N-terminal fragment of an LLO protein comprises SEQ ID NO: 2.

3. The immunogenic composition of any one of claims 1-2, wherein said recombinant Listeria strain is a recombinant Listeria monocytogenes strain.

4. The immunogenic composition of any one of claims 1-3, wherein, wherein said recombinant polypeptide is expressed by said recombinant Listeria strain.

5. The immunogenic composition of any one of claims 1-4, wherein said nucleic acid is in an extrachromosomal plasmid in said recombinant Listeria strain.

6. The immunogenic composition of any one of claims 1-5, wherein said plasmid is stably maintained in said recombinant Listeria strain.

7. The immunogenic composition of any one of claims 1-6, wherein said Listeria strain comprises a mutation, deletion or inactivation in the genomic dal, dat, and actA genes.

8. The immunogenic composition of any one of claims 1-7, wherein said metabolic enzyme complements said mutation, deletion or inactivation.

9. The immunogenic composition of claim 1, wherein a structure of said recombinant polypeptide encoded by said first open reading frame comprises an N-terminal LLO-antigen 1-antigen 2-antigen 3-antigen 4.

10. The immunogenic composition of any one of claims 1-9, wherein said four heterologous HPV antigens or functional fragment thereof are selected from the group consisting of HPV16 E6, HPV16 E7, HPV18 E6, HPV18 E7 antigens, and fragments thereof.

11. The immunogenic composition of claim 9, wherein antigen 1 is HPV16 E6, antigen 2 is HPV16 E7, antigen3 is HPV18 E6 and antigen 4 is HPV18 E7.

12. The immunogenic composition of any one of claims 1-11, wherein said first open reading frame further encodes a protein tag sequence operably linked at the C-terminus of the last heterologous antigen.

13. The immunogenic composition of claim 12, wherein said protein tag sequence is SIINFEKL-6xHIS sequence.

14. The immunogenic composition of any one of claims 12-13, wherein said first open reading frame comprises SEQ ID NO: 32.

15. The immunogenic composition of any one of claims 12-13, wherein said first open reading frame encodes a protein comprising SEQ ID NO: 33.

16. The immunogenic composition of any one of claims 1-15, wherein said recombinant Listeria strain has been passaged through an animal host.

17. The immunogenic composition of any one of claims 1-16, further comprising an adjuvant.

18. The immunogenic composition of any one of claim 17, wherein said adjuvant is selected from the list comprising a GM-CSF protein, saponin QS21, monophosphoryl lipid A, SBAS2, an unmethylated CpG-containing oligonucleotide, an immune-stimulating cytokine, a quill glycoside, a bacterial toxin, and a bacterial mitogen.

19. The immunogenic composition of any one of claims 1-18, wherein said recombinant Listeria strain has been stored in a frozen or lyophilized cell bank.

20. A method of treating tumor or cancer in a subject, the method comprising the step of administering to said subject an immunogenic composition of any one of claims 1-19.

21. A method of protecting a human subject against a tumor or cancer, comprising the step of administering to said subject the composition of any one of claims 1-19.

22. A method for inducing an anti-tumor cytotoxic T cell response in a human subject, comprising the step of administering to said subject a composition comprising a recombinant Listeria strain of any one claims 1-19.

23. The method of any one of claims 20-22, wherein the composition is administered at a dose of about 1×10⁶ CFU to about 1×10⁹ CFU.

24. The method of claim 23, wherein the composition is administered at a dose of about 1×10⁶ CFU to about 1×10⁸ CFU.

25. The method of claim 23, wherein the composition is administered at a dose of about 1×10⁶ CFU to about 1×10⁷ CFU.

26. The method of claim 23, wherein the composition is administered at a dose of about 1×10⁷ CFU to about 1×10⁹ CFU.
